(19) United States
(12) Patent Application Publication
(10) Pub. No.: US 2023/0113811 A1
(43) Pub. Date: Apr. 13, 2023

(54) SYSTEMS AND METHODS TO PROCESS ELECTRONIC IMAGES TO IDENTIFY MUTATIONAL SIGNATURES AND TUMOR SUBTYPES

(71) Applicant: PAIGE.AI, Inc., New York, NY (US)

(72) Inventors: Yikan WANG, Burnaby (CA); Christopher KANAN, Pittsford, NY (US); Patricia RACITI, New York, NY (US)

(21) Appl. No.: 17/956,066

(22) Filed: Sep. 29, 2022

Related U.S. Application Data
(60) Provisional application No. 63/254,551, filed on Oct. 12, 2021.

(51) Int. Cl. G16H 30/40; G16H 50/20

(52) U.S. Cl. CPC G16H 30/40; G16H 50/20

(57) ABSTRACT

A method for identifying a mutational signature may include receiving one or more digital images into electronic storage for at least one patient, identifying one or more neoplasms in each received digital image, extracting one or more visual features from each identified neoplasm, and applying a trained machine learning system to identify a mutational signature ratio vector for the one or more extracted visual features.

### RELATED APPLICATION(S)

This application claims priority to U.S. Provisional Application No. 63/254,551 filed Oct. 12, 2021, the entire disclosure of which is hereby incorporated herein by reference in its entirety.

### FIELD OF THE DISCLOSURE

Various embodiments of the present disclosure pertain generally to image processing methods. More specifically, particular embodiments of the present disclosure relate to systems and methods to identify mutational signatures and tumor subtypes.

### BACKGROUND

Cancers consist of multiple mutations, and distinct mutation combinations may arise based on specific mutagens. These are known as mutational signatures.

Somatic mutations in cancer genomes are the consequences of multiple mutational processes. Mutational signatures are characteristic combinations of mutation types arising from specific mutagenesis processes, such as deoxyribonucleic acid (DNA) replication infidelity, exogenous (well-known mutagens, such as ultraviolet or UV radiation and tobacco smoke) and endogenous genotoxins (such as those involving activated DNA cytidine deaminases/apolipoprotein B editing complex, activation-induced cytidine deaminase/apolipoprotein B mRNA-editing enzyme catalytic polypeptide-like enzymes or proteins, or AID/APOBECs) exposures, defective DNA repair pathways, and DNA enzymatic editing.

There are mutational signatures from whole genome sequencing or exome sequencing data, characterizing the mutational processes across the spectrum of human cancer.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the present disclosure, systems and methods are disclosed for a computer-implemented method for identifying a mutational signature. The method may include receiving one or more digital images into electronic storage for at least one patient, identifying one or more neoplasms in each received digital image, extracting one or more visual features from each identified neoplasm, and applying a trained machine learning system to identify a mutational signature ratio vector for the one or more extracted visual features.

The extracted visual features may include neoplasm embeddings. Identifying one or more neoplasms may include segmenting each received digital image into subregions.

The method may further include determining, for each identified mutational signature ratio vector, whether a largest value in the mutational signature ratio vector is below a predetermined certainty threshold. The method may further include determining that the largest value in the mutational signature ratio is below the predetermined certainty threshold. The method may include determining that a mutational signature corresponding to the mutational signature ratio is unknown.

Receiving one or more digital images for at least one patient may include receiving a plurality of digital images for a plurality of patients. Applying the trained machine learning system to identify the mutational signature ratio vector may include identifying a plurality of mutational signature ratio vectors. The method may include identifying a set of patients among the plurality of patients that have an unknown mutational signature.

The method may include clustering extracted visual features for the identified set of patients. The method may include receiving patient information for each patient among the plurality of patients. The method may include determining, based on the received patient information and clustered extracted visual features, whether any of the unknown signatures are associated with mutagens.

Receiving one or more digital images for at least one patient may include receiving a plurality of digital images for a plurality of patients. Applying the trained machine learning system to identify the mutational signature ratio vector may include identifying a plurality of mutational signature ratio vectors. The method may further include receiving patient information for each patient among the plurality of patients, determining a set of patients who have similar clinical phenotypes, and determining disease subtypes based on the identified mutational signature ratio vectors of the determined set of patients.

Receiving one or more digital images for at least one patient may include receiving a plurality of digital images for a plurality of patients. Applying the trained machine learning system to identify the mutational signature ratio vector may include identifying a plurality of mutational signature ratio vectors. The method may further include receiving treatment information for each patient among the plurality of patients, and training a machine learning system that predicts a treatment response based on the identified mutational signature ratio vectors and received treatment information.

Receiving one or more digital images for at least one patient may include receiving a plurality of digital images for a plurality of patients. Applying the trained machine learning system to identify the mutational signature ratio vector may include identifying a plurality of mutational signature ratio vectors. The method may further include receiving patient information for each of the plurality of patients, receiving an indication of a geographic location of each patient, and determining, based on the received indications of the geographic locations, whether any of the mutational signature ratio vectors are associated with certain geographic locations.

Receiving one or more digital images for at least one patient may include receiving a plurality of digital images for a plurality of patients. Applying the trained machine learning system to identify the mutational signature ratio vector may include identifying a plurality of mutational signature ratio vectors. The method may further include clustering extracted visual features for the plurality of patients and determining a mutational signature ratio vector among the identified mutational signature ratio vectors correspond to an unknown mutagen.

According to certain aspects of the present disclosure, systems and methods are disclosed for processing electronic images. A system may for processing electronic images may include at least one memory storing instructions and at least one processor configured to execute the instructions to perform operations. The operations may include receiving one or more digital images into electronic storage for at least one patient, identifying one or more neoplasms in each received digital image, extracting one or more visual features from each identified neoplasm, and applying a trained machine learning system to identify a mutational signature ratio vector for the one or more extracted visual features.

The operations may further include determining, for each identified mutational signature ratio vector, whether a largest value in the mutational signature ratio vector is below a predetermined certainty threshold. The operations may further include determining that the largest value in the mutational signature ratio is below the predetermined certainty threshold and determining that a mutational signature corresponding to the mutational signature ratio is unknown.

Receiving one or more digital images for at least one patient may include receiving a plurality of digital images for a plurality of patients. Applying the trained machine learning system to identify the mutational signature ratio vector may include identifying a plurality of mutational signature ratio vectors. The operations may further include identifying a set of patients among the plurality of patients that have an unknown mutational signature.

The operations may further include clustering extracted visual features for the identified set of patients. The operations may further include receiving patient information for each patient among the plurality of patients and determining, based on the received patient information and clustered extracted visual features, whether any of the unknown signatures are associated with mutagens.

According to certain aspects of the present disclosure, systems and methods are disclosed for processing electronic images. A non-transitory computer-readable medium may store instructions that, when executed by a processor, perform operations processing electronic medical images. The operations may include receiving one or more digital images into electronic storage for at least one patient, identifying one or more neoplasms in each received digital image, extracting one or more visual features from each identified neoplasm, and applying a trained machine learning system to identify a mutational signature ratio vector for the one or more extracted visual features. The operations may include determining, for each identified mutational signature ratio vector, whether a largest value in the mutational signature ratio vector is below a predetermined certainty threshold.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A illustrates an exemplary display of the six substitution subtypes and a calculation of mutations per megabase (mutations/MB).

FIB. 1B shows an exemplary series of frequency charts illustrating the six substitution subtypes of FIG. 1A, signatures, and contributions.

FIG. 1C shows the mutational activities of FIG. 1B of corresponding mutational signatures displayed as a pie chart.

FIG. 2 illustrates exemplary compositions of mutation signatures across various cancer types.

FIGS. 3A and 3B illustrate exemplary known and unknown signatures.

FIG. 4A illustrates an exemplary block diagram of a system and network to synthetically augment or modify digital or electronic slide images to change slide quality, according to an exemplary embodiment of the present disclosure.

FIG. 4B illustrates an exemplary block diagram of a disease detection platform, according to an exemplary embodiment of the present disclosure.

FIG. 4C illustrates an exemplary block diagram of a slide analysis tool, according to an exemplary embodiment of the present disclosure.

FIG. 5 is an exemplary flow chart illustrating a process for training a neoplasm detection module according to an exemplary embodiment of the present disclosure.

FIG. 6 is an exemplary flow chart illustrating a process for using a neoplasm detection module according to an exemplary embodiment of the present disclosure.

FIG. 7 is an exemplary flow chart illustrating a process for training a signature inference module according to an exemplary embodiment of the present disclosure.

FIG. 8 is an exemplary flow chart illustrating a process for using a signature inference module according to an exemplary embodiment of the present disclosure.

FIG. 9 is an exemplary flow chart illustrating a process for determining a cancer subtype according to an exemplary embodiment of the present disclosure.

FIG. 10 is an exemplary flow chart illustrating a process for training a system to recommend treatment according to an exemplary embodiment of the present disclosure.

FIG. 11 is an exemplary flow chart illustrating a process for using a system to recommend treatment according to an exemplary embodiment of the present disclosure.

FIG. 12 is an exemplary flow chart illustrating a process for identifying new mutational signatures according to an exemplary embodiment of the present disclosure.

FIG. 13 is an exemplary flow chart illustrating a process for identifying unknown mutagens according to an exemplary embodiment of the present disclosure.

FIG. 14 is an exemplary flow chart illustrating a process for identifying a geographic location specific signature according to an exemplary embodiment of the present disclosure.

FIG. 15 is an exemplary flow chart illustrating a process for determining a measure of generalization, according to an exemplary embodiment of the present disclosure.

### DESCRIPTION OF THE EMBODIMENTS

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Genome-wide sequencing data analyses identify signatures from different genomic variant classes associated with various exposures, including copy number signatures. A repertoire of mutational signatures derived from single base substitutions, double base substitution, and small insertions and deletions have been established.

Some approaches of extracting signatures from these genomic classes include non-negative matrix factorization (NMF), which is an unsupervised machine learning method. Although these approaches provide a selected number of patterns without requiring any prior knowledge of the endogenous and exogenous factors, the patterns are retrospectively correlated with previously known mutation patterns or clinical data to reveal potential mutational processes underlying the signatures. Other than the well-known mutational signatures, however the causes of many signatures, their underlying mechanisms, and the clinical implications remain unknown.

There is a wide-association between histopathological patterns and a broad range of genetic aberrations including copy number variants, driver gene mutations, as well as gene expression profiles. However, how histopathological features are associated with mutation processes have not been previously defined.

Techniques presented herein describe systems and methods which may determine what mutation signatures are present in a patient's tissue and may identify new signatures. Systems and methods disclosed herein may guide treatment, gain insights into the causes of cancer, and identify rare tumor subtypes. Systems and methods disclosed herein may infer known and unknown mutational signatures from digital medical images.

Techniques presented herein may use artificial intelligence (AI) to track mutation signatures in a tissue sample, identify new signatures, and identify rare tumor subtypes. Techniques presented herein may use AI to infer or determine known and unknown mutational signatures from whole slide images (WSIs) of issue samples or other samples.

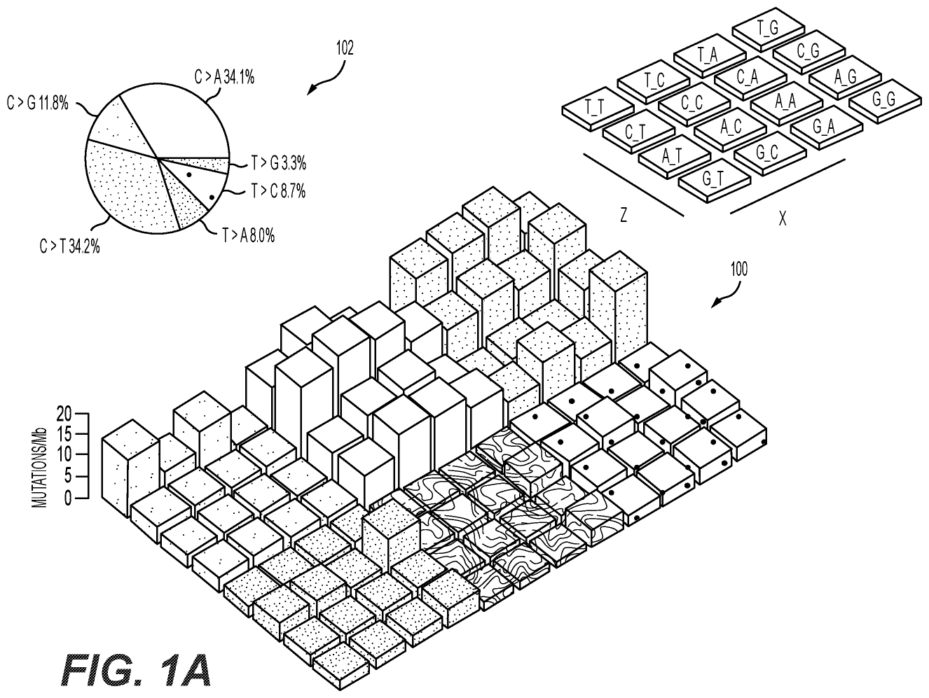

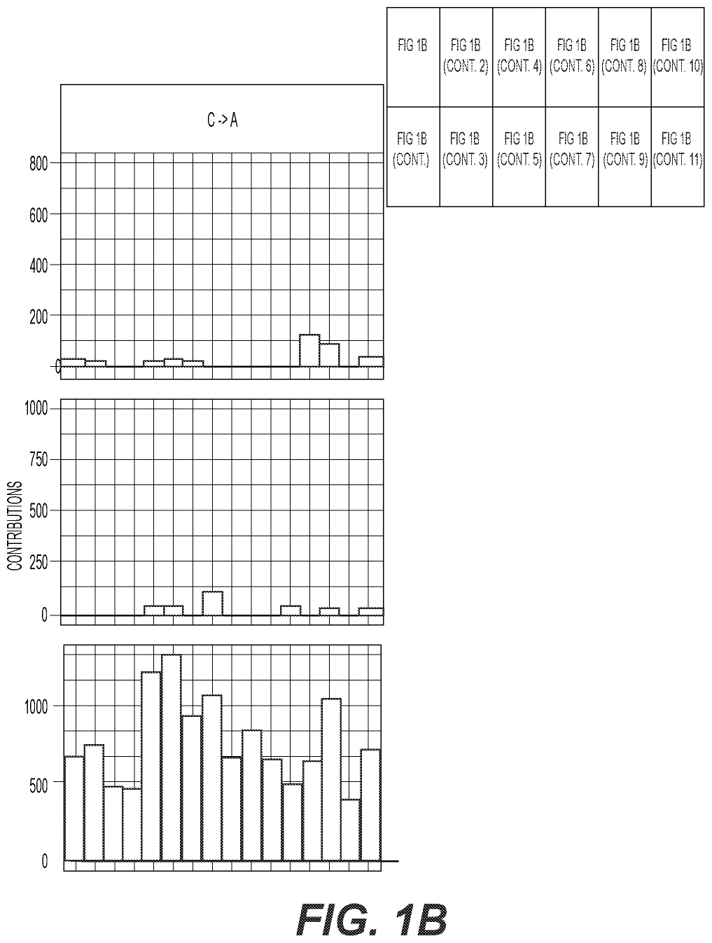

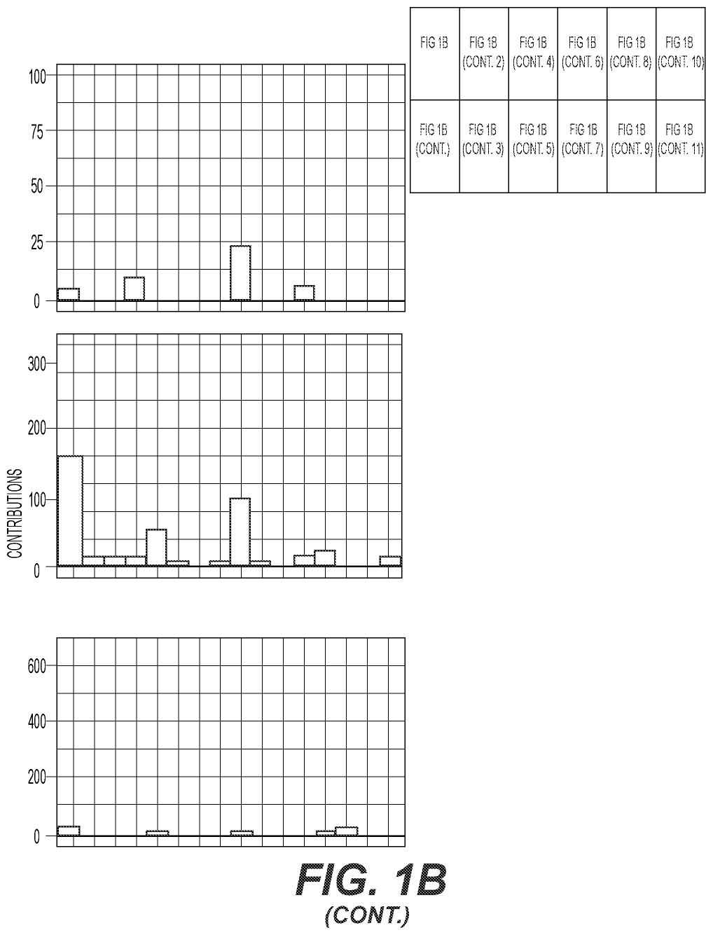

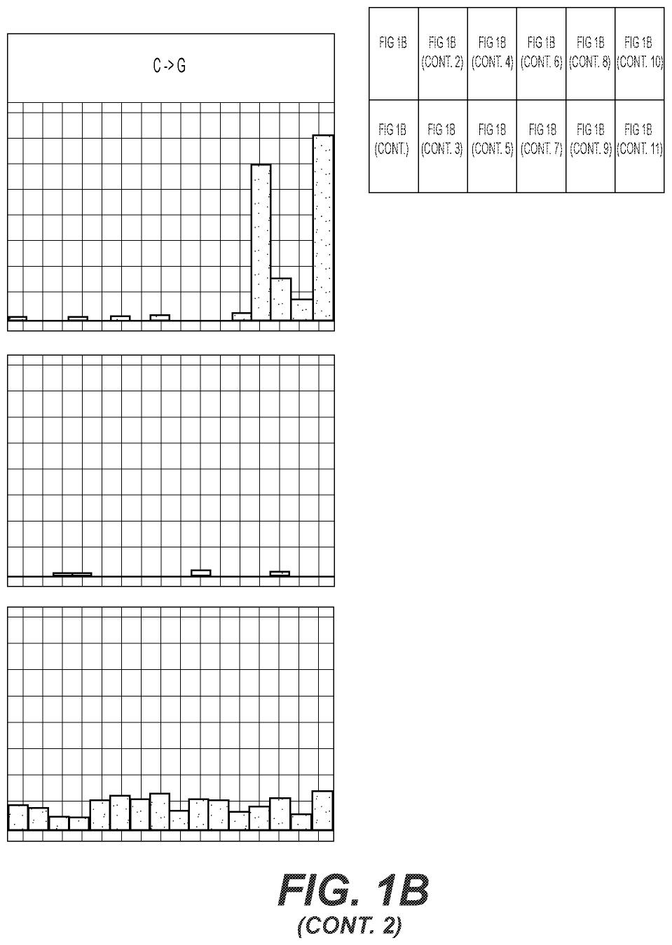

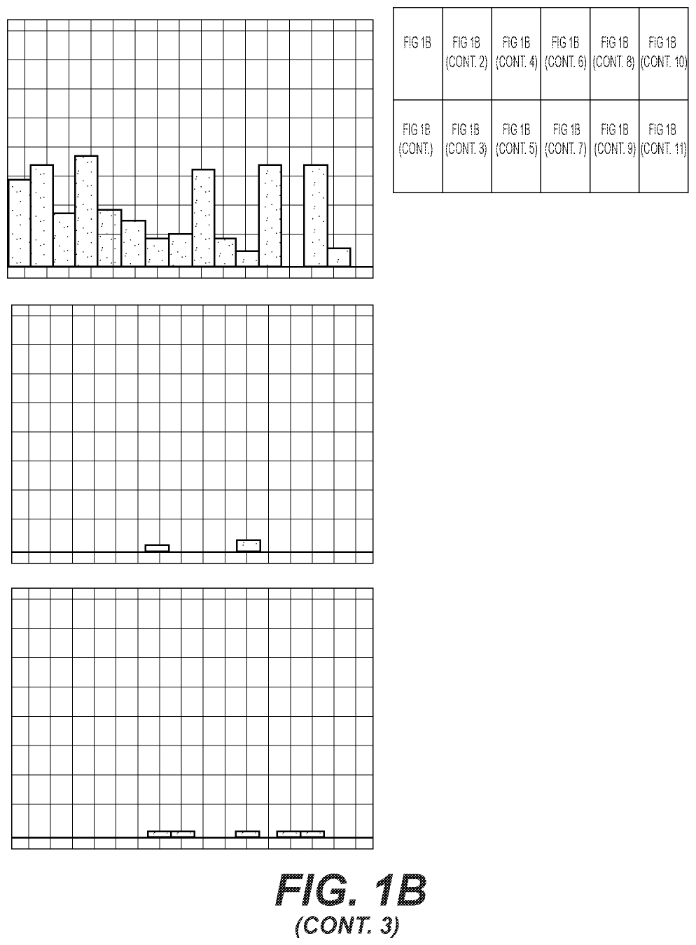

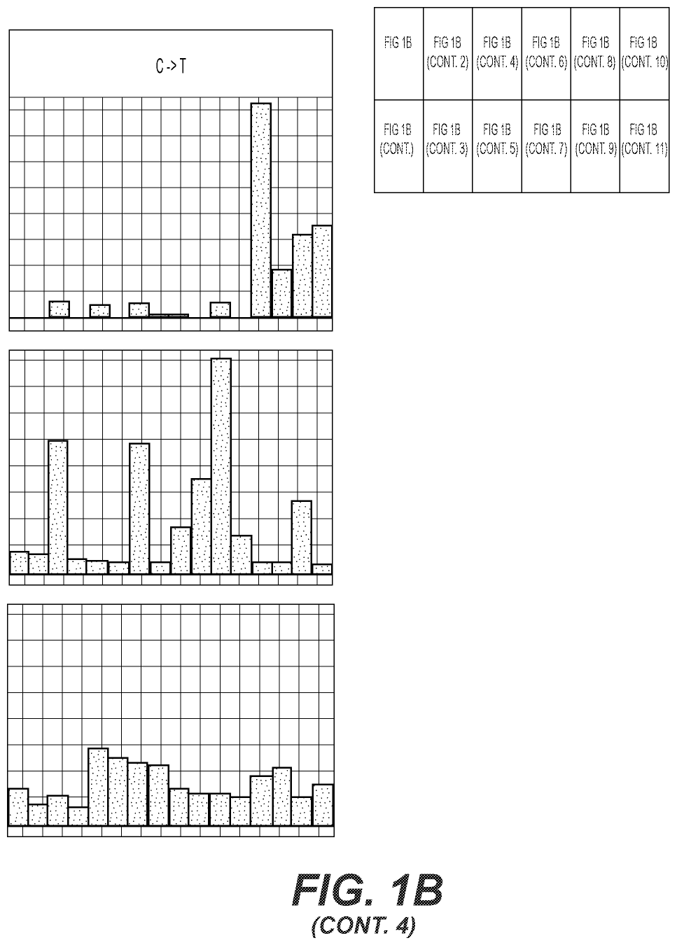

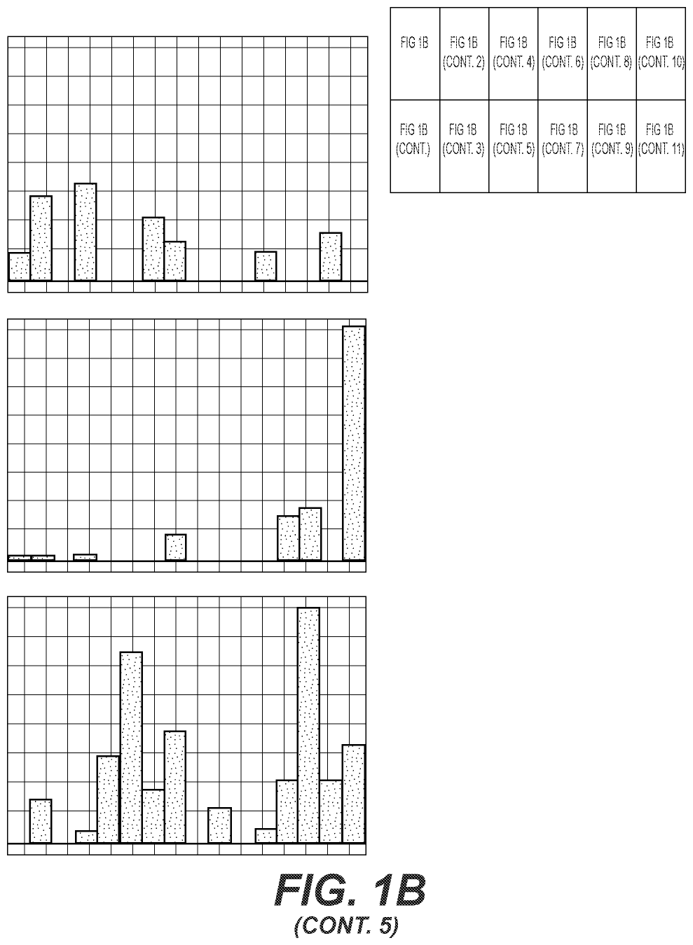

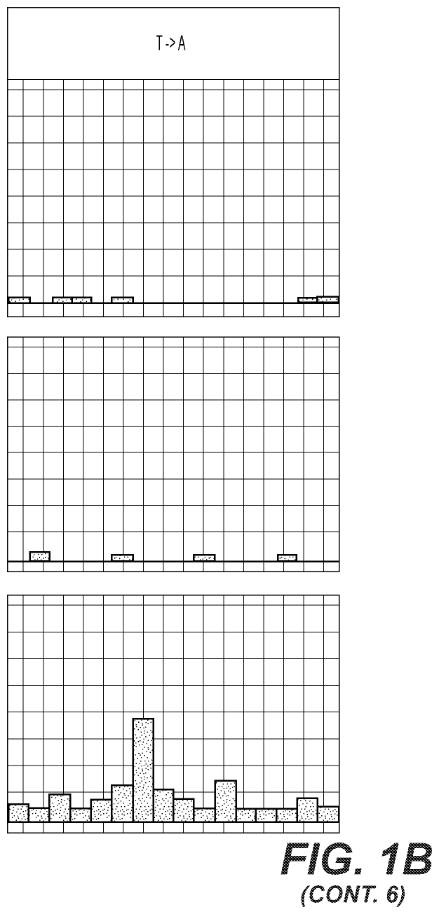

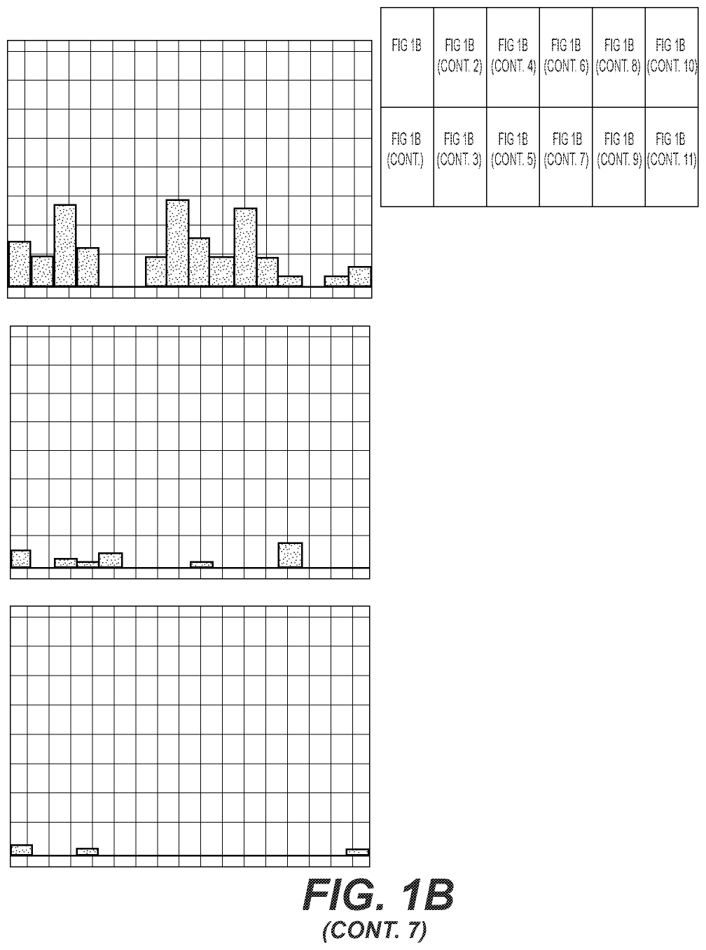

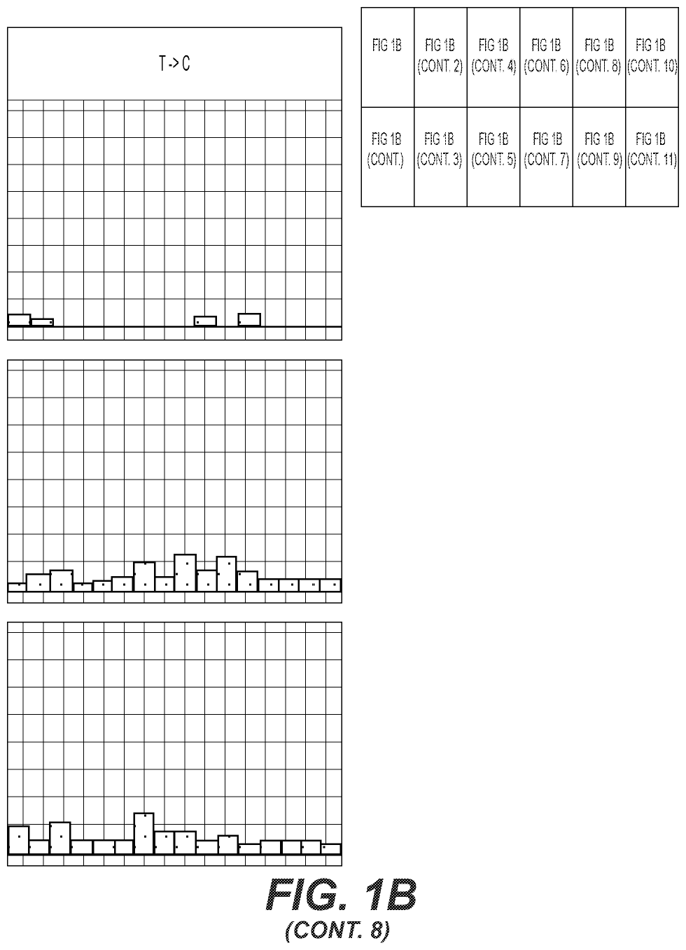

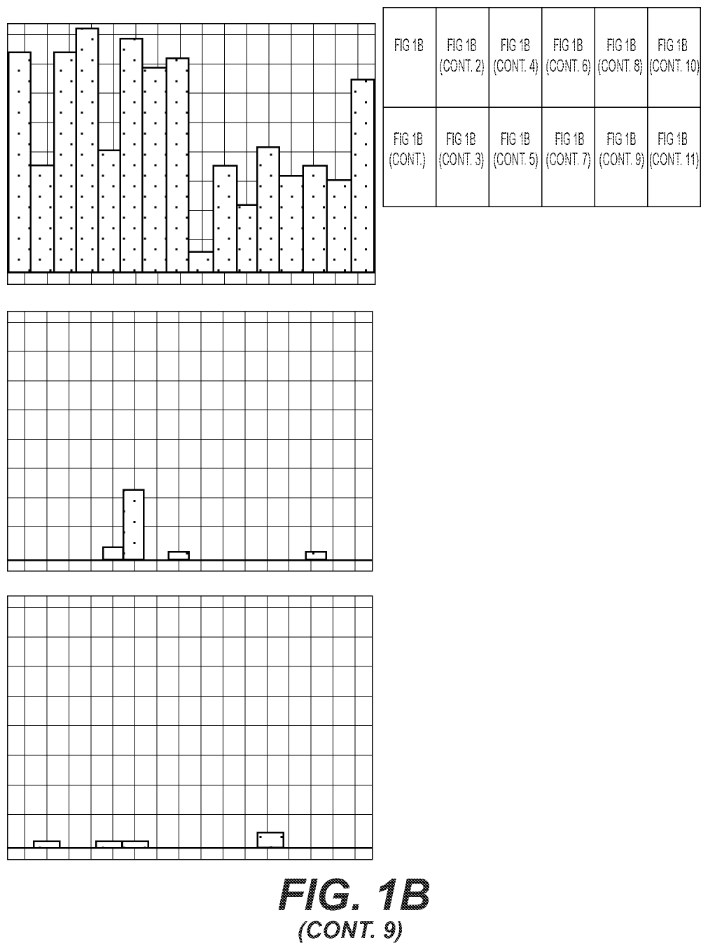

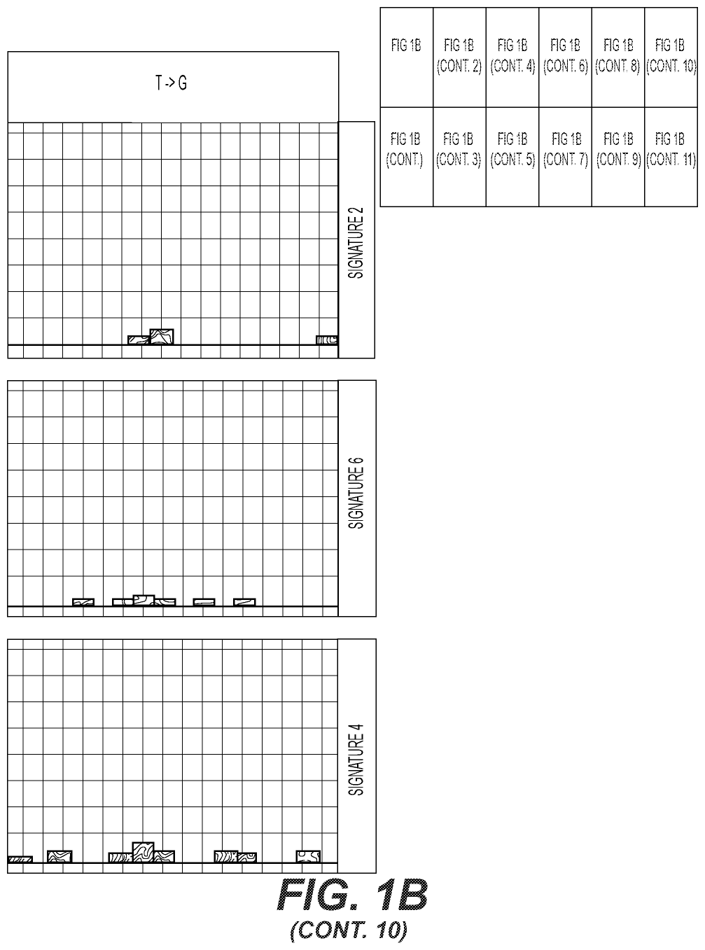

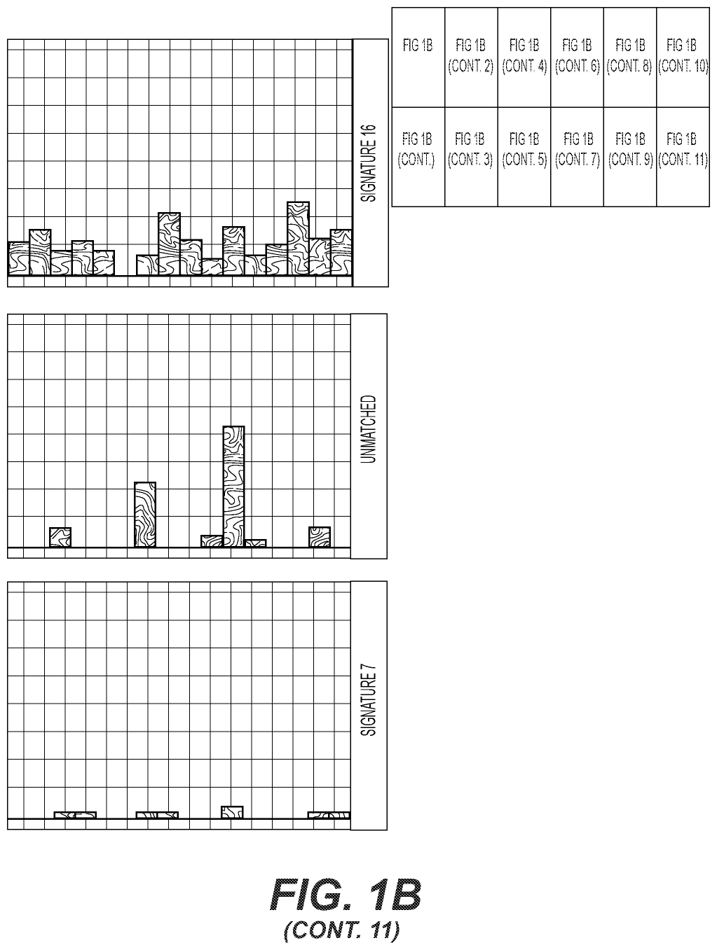

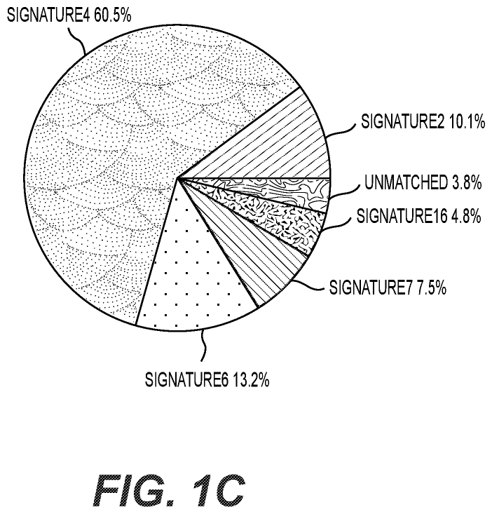

Referring to FIGS. 1A, 1B, and 1C, analyses may reveal mutational signatures from whole genome sequencing or exome sequencing data, characterizing mutational processes across the spectrum of human cancer. These mutation signatures may be displayed using six substitution subtypes: C>A, C>G, C>T, T>A, T>C, T>G (FIG. 1A), and computed from the frequency of trinucleotide substitution patterns in human genome (FIGS. 1B and 1C). Retrospective analysis may be applied to reveal what potential mutation processes might be associated with these signatures.

FIG. 1A shows an exemplary display of the six substitution subtypes and a calculation of mutations per megabase (mutations/MB). A lego plot 100 may represent mutation patterns in 113 non-small cell lung cancer (NSCLC) samples. Single-nucleotide substitutions may be divided into the six substitution subtypes with 16 surrounding flanking bases. A pie chart 102 may show a proportion of the subtypes of mutation patterns.

FIB. 1B shows an exemplary series of frequency charts illustrating the six substitution subtypes, signatures, and contributions. In particular, FIG. 1B shows the mutational activities of corresponding extracted mutational signatures (signatures 2, 4, 6, 7, 16, 26, and unmatched). FIG. 1C shows the mutational activities of corresponding mutational signatures displayed as a pie chart.

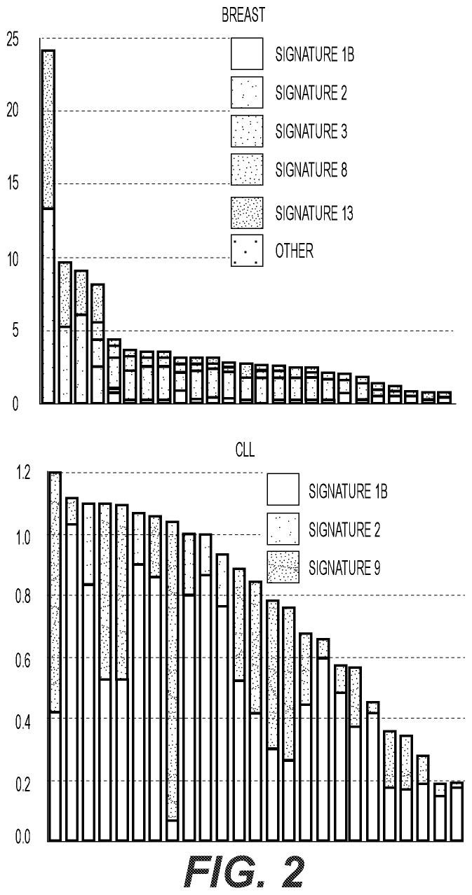

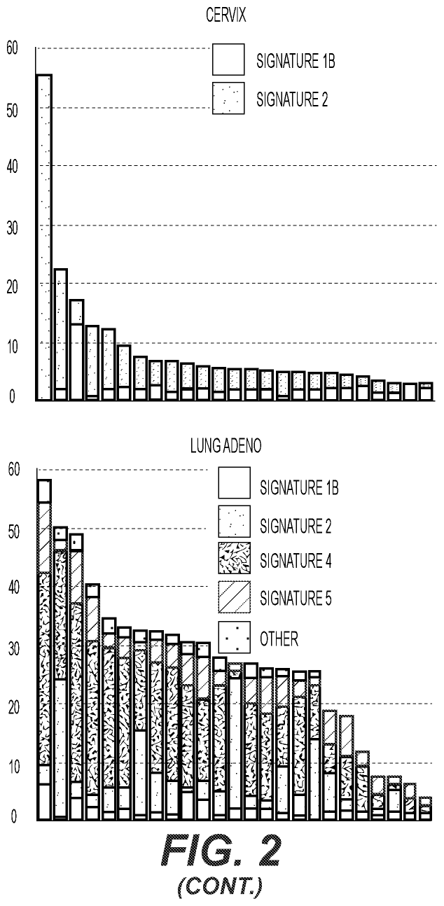

Referring to FIG. 2, a composition of mutation signatures varies across cancer types. FIG. 2 depicts a bar graph for each cancer type. Each bar may represent a typical selected sample from the respective cancer type, and the vertical axis may denote the number of mutations per megabase. An age of a cancer diagnosis signature may be seen in most cancer types. Some mutational signatures may be associated with failures in DNA repair mechanisms, e.g., homologous recombination deficiency (HRD) and mismatch repair defects (MMR). HRD and MMR signatures may be highly associated with breast, ovarian, and endometrial cancers, while a tobacco smoking-associated signature may be highly associated with lung, liver, and head and neck cancers. A UV light exposure associated signature may be prominent in skin cancer.

These signatures may be useful biomarkers for a range of cancer types and may provide implications for pathogenesis and diagnosis. These signatures may be useful biomarkers for identifying patients who might have better treatment response to chemotherapy or who might develop chemo-resistance and for suggesting targeted therapies for effective treatment strategies.

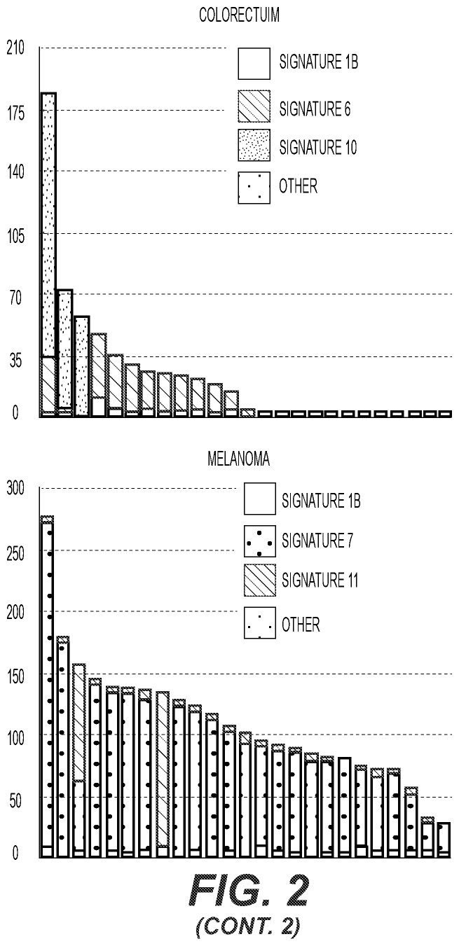

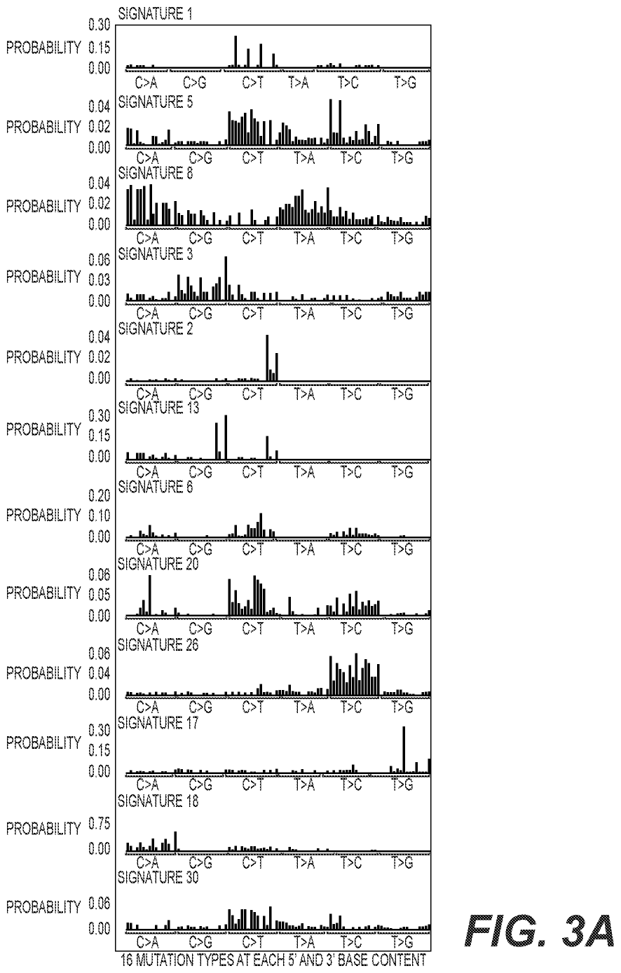

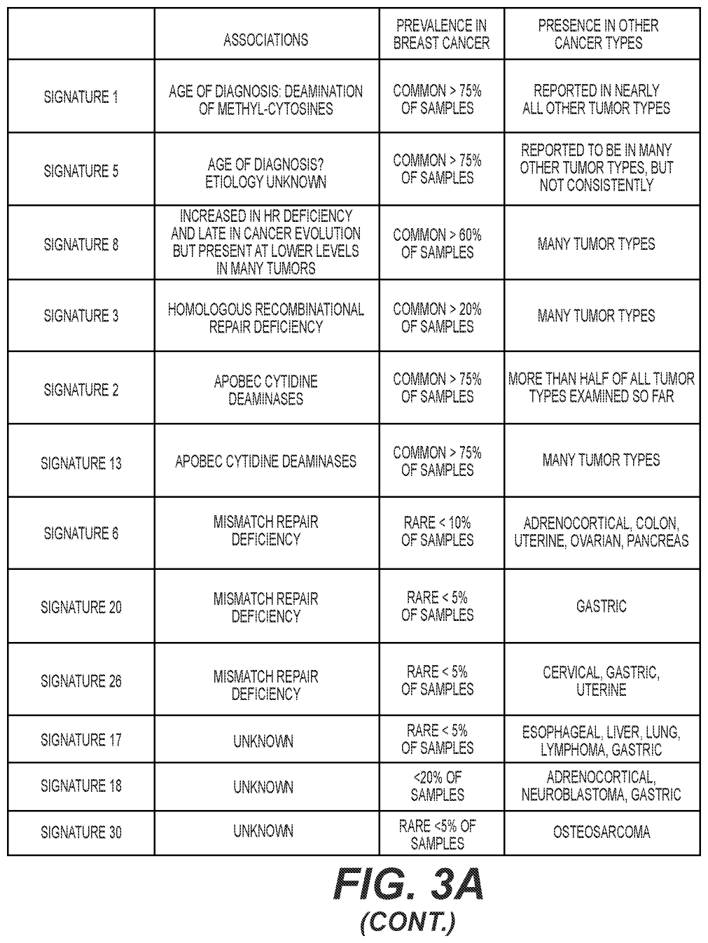

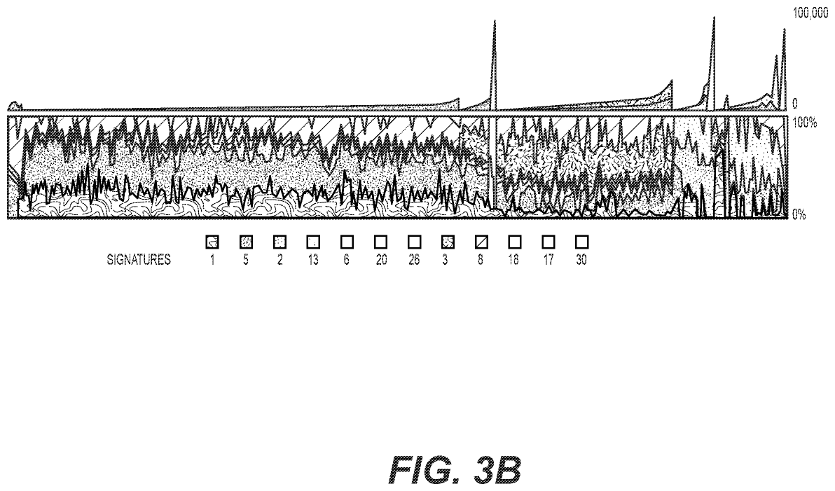

There are a number of signatures from different genomic variant classes associated with various exposures, including copy number signatures and genome rearrangement signatures, and there are a repertoire of mutational signatures derived from single base substitutions, double base substitution and small insertions and deletions. Referring to FIGS. 3A and 3B, other than some known mutational signatures, however the causes of many signatures, their underlying mechanisms, and the clinical implications remain unknown. There is a wide association between histopathological patterns and a broad range of genetic aberrations including copy number variants, driver gene mutations, and gene expression profiles; however, how histopathological features are associated with mutation processes are not yet defined.

Methods and systems disclosed herein may provide a system to infer mutational signatures and discover new signatures from hematoxylin and eosin (H&E) stained histological whole slide images (WSI). Methods and systems disclosed herein may correlate computational learned histological features to well-known mutational signatures, and link any novel signatures to morphology, mutation patterns, DNA methylation, proteomic profiling, immune infiltration, treatment response.

Systems and methods disclosed herein may have two primary components. The first may be a “Neoplasm Detection Module” to detect all neoplasms on a slide. The second may be a “Signature Inference Module” to infer signatures present and flag unknown signatures. The signature inference module may be capable of outputting the signatures present from digital images as either a list or as a spatially organized representation. Systems and methods disclosed herein may operate on digital medical images, which could be whole slide images (WSIs) of pathology data (multiplex, RGB, etc.), radiology scans, etc.

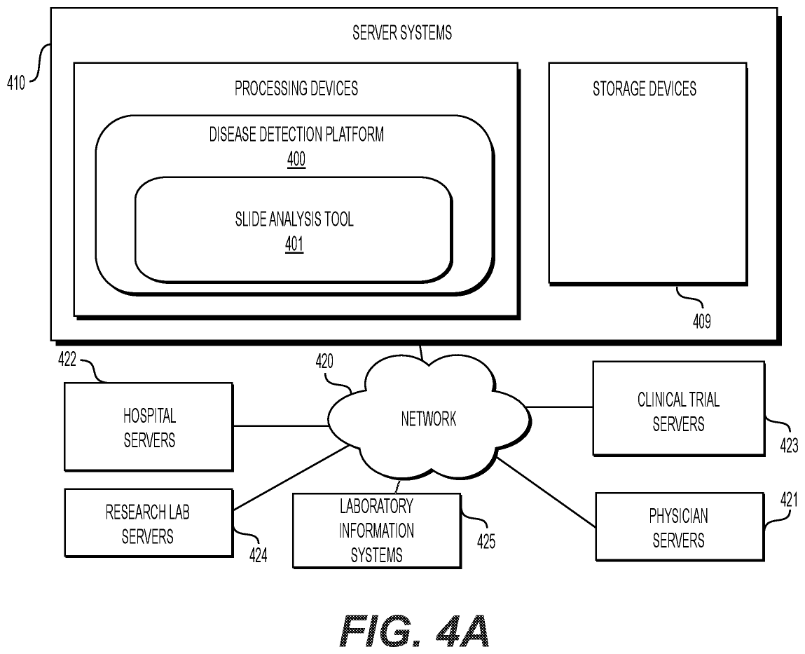

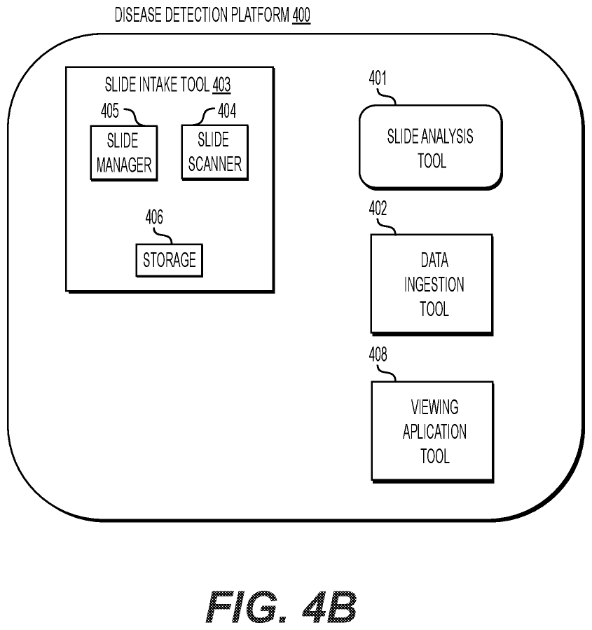

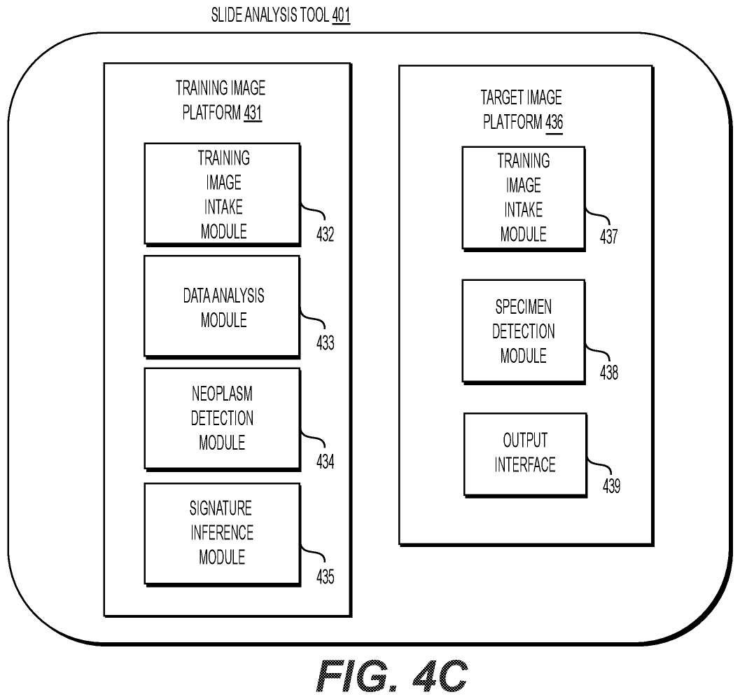

Referring to FIGS. 4A, 4B, and 4C, FIGS. 4A through 4C show a system and network to identify mutational signatures and tumor subtypes, according to an exemplary embodiment of the present disclosure.

Specifically, FIG. 4A illustrates an electronic network 120 that may be connected to servers at hospitals, laboratories, and/or doctor's offices, etc. For example, physician servers 421, hospital servers 422, clinical trial servers 423, research lab servers 424, and/or laboratory information systems 425, etc., may each be connected to an electronic network 420, such as the Internet, through one or more computers, servers and/or handheld mobile devices. According to an exemplary embodiment of the present application, the electronic network 420 may also be connected to server systems 410, which may include processing devices that are configured to implement a disease detection platform 400, which includes a slide analysis tool 401 for determining specimen property or image property information pertaining to digital pathology image(s), and using machine learning to determine whether a disease or infectious agent is present, according to an exemplary embodiment of the present disclosure. The slide analysis tool 401 may allow for rapid evaluation of ‘adequacy’ in liquid-based tumor preparations, facilitate the diagnosis of liquid based tumor preparations (cytology, hematology/hematopathology), and predict molecular findings most likely to be found in various tumors detected by liquid-based preparations.

The physician servers 421, hospital servers 422, clinical trial servers 423, research lab servers 424 and/or laboratory information systems 425 may create or otherwise obtain images of one or more patients' cytology specimen(s), histopathology specimen(s), slide(s) of the cytology specimen(s), digitized images of the slide(s) of the histopathology specimen(s), or any combination thereof. The physician servers 421, hospital servers 422, clinical trial servers 423, research lab servers 424 and/or laboratory information systems 425 may also obtain any combination of patient-specific information, such as age, medical history, cancer treatment history, family history, past biopsy or cytology information, etc. The physician servers 421, hospital servers 422, clinical trial servers 423, research lab servers 424 and/or laboratory information systems 425 may transmit digitized slide images and/or patient-specific information to server systems 410 over the electronic network 420. Server system(s) 410 may include one or more storage devices 409 for storing images and data received from at least one of the physician servers 421, hospital servers 422, clinical trial servers 423, research lab servers 424, and/or laboratory information systems 425. Server systems 410 may also include processing devices for processing images and data stored in the storage devices 409. Server systems 410 may further include one or more machine learning tool(s) or capabilities. For example, the processing devices may include a machine learning tool for a disease detection platform 400, according to one embodiment. Alternatively or in addition, the present disclosure (or portions of the system and methods of the present disclosure) may be performed on a local processing device (e.g., a laptop).

The physician servers 421, hospital servers 422, clinical trial servers 423, research lab servers 424 and/or laboratory information systems 425 refer to systems used by pathologists for reviewing the images of the slides. In hospital settings, tissue type information may be stored in a laboratory information system 425.

FIG. 4B illustrates an exemplary block diagram of a disease detection platform 400 for determining specimen property or image property information pertaining to digital pathology image(s), using machine learning. The disease detection platform 400 may include a slide analysis tool 401, a data ingestion tool 402, a slide intake tool 403, a slide scanner 404, a slide manager 405, a storage 406, and a viewing application tool 408.

The slide analysis tool 401, as described below, refers to a process and system for determining data variable property or health variable property information pertaining to digital pathology image(s). Machine learning may be used to classify an image, according to an exemplary embodiment. The slide analysis tool 401 may also predict future relationships, as described in the embodiments below.

The data ingestion tool 402 may facilitate a transfer of the digital pathology images to the various tools, modules, components, and devices that are used for classifying and processing the digital pathology images, according to an exemplary embodiment.

The slide intake tool 403 may scan pathology images and convert them into a digital form, according to an exemplary embodiment. The slides may be scanned with slide scanner 404, and the slide manager 405 may process the images on the slides into digitized pathology images and store the digitized images in storage 406.

The viewing application tool 408 may provide a user with a specimen property or image property information pertaining to digital pathology image(s), according to an exemplary embodiment. The information may be provided through various output interfaces (e.g., a screen, a monitor, a storage device and/or a web browser, etc.).

The slide analysis tool 401, and one or more of its components, may transmit and/or receive digitized slide images and/or patient information to server systems 410, physician servers 421, hospital servers 422, clinical trial servers 423, research lab servers 424, and/or laboratory information systems 425 over a network 420. Further, server systems 410 may include storage devices for storing images and data received from at least one of the slide analysis tool 401, the data ingestion tool 402, the slide intake tool 403, the slide scanner 404, the slide manager 405, and viewing application tool 408. Server systems 410 may also include processing devices for processing images and data stored in the storage devices. Server systems 410 may further include one or more machine learning tool(s) or capabilities, e.g., due to the processing devices. Alternatively, or in addition, the present disclosure (or portions of the system and methods of the present disclosure) may be performed on a local processing device (e.g., a laptop).

Any of the above devices, tools, and modules may be located on a device that may be connected to an electronic network such as the Internet or a cloud service provider, through one or more computers, servers and/or handheld mobile devices.

FIG. 4C illustrates an exemplary block diagram of a slide analysis tool 101, according to an exemplary embodiment of the present disclosure. The slide analysis tool 401 may include a training image platform 431 and/or a target image platform 436.

According to one embodiment, the training image platform 431 may include a training image intake module 432, a data analysis module 433, a neoplasm detection module 434, and a signature inference module 435. Alternatively or in addition thereto, the neoplasm detection module 434 and the signature inference module 435 may be combined as one module (e.g., a signature identification module) and/or may be included in slide intake tool 403 or as part of data ingestion tool 402.

The training data platform 431, according to one embodiment, may create or receive training images that are used to train a machine learning model to effectively analyze and classify digital pathology images. For example, the training images may be received from any one or any combination of the server systems 410, physician servers 421, hospital servers 422, clinical trial servers 423, research lab servers 424, and/or laboratory information systems 425. Images used for training may come from real sources (e.g., humans, animals, etc.) or may come from synthetic sources (e.g., graphics rendering engines, 3D models, etc.). Examples of digital pathology images may include (a) digitized slides stained with a variety of stains, such as (but not limited to) H&E, Hematoxylin alone, IHC, molecular pathology, etc.; and/or (b) digitized tissue samples from a 3D imaging device, such as microCT.

The training image intake module 432 may create or receive a dataset comprising one or more training datasets corresponding to one or more health variables and/or one or more data variables. For example, the training datasets may be received from any one or any combination of the server systems 410, physician servers 421, hospital servers 422, clinical trial servers 423, research lab servers 424, and/or laboratory information systems 425. This dataset may be kept on a digital storage device. The data analysis module 433 may identify whether an area belongs to a region of interest or salient region, or to a background of a digitized image.

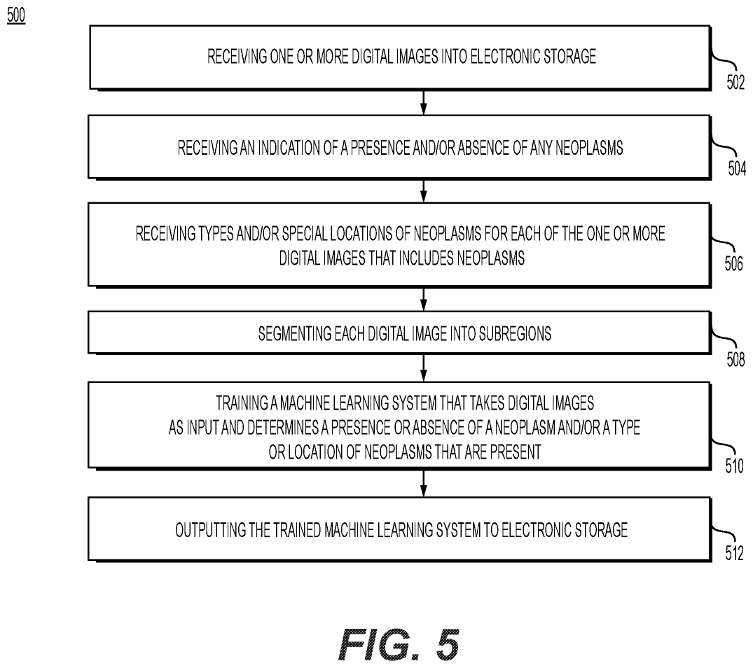

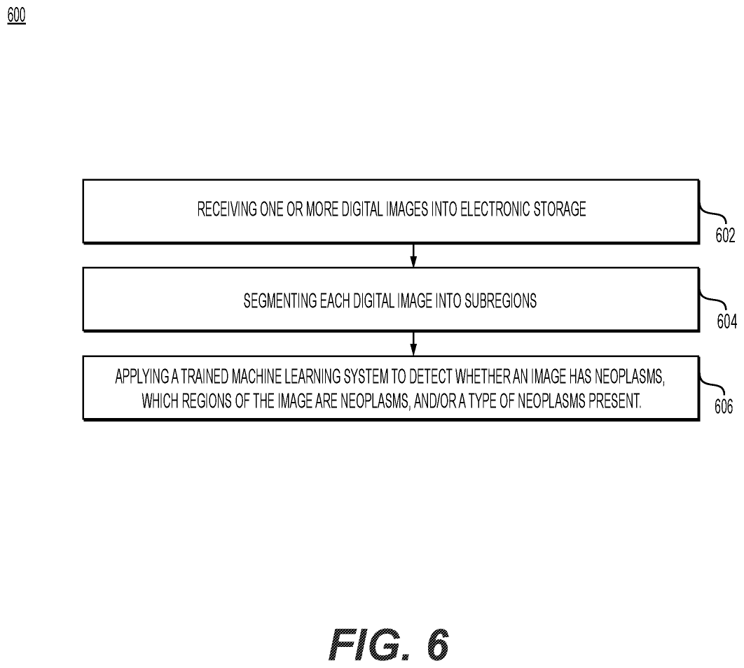

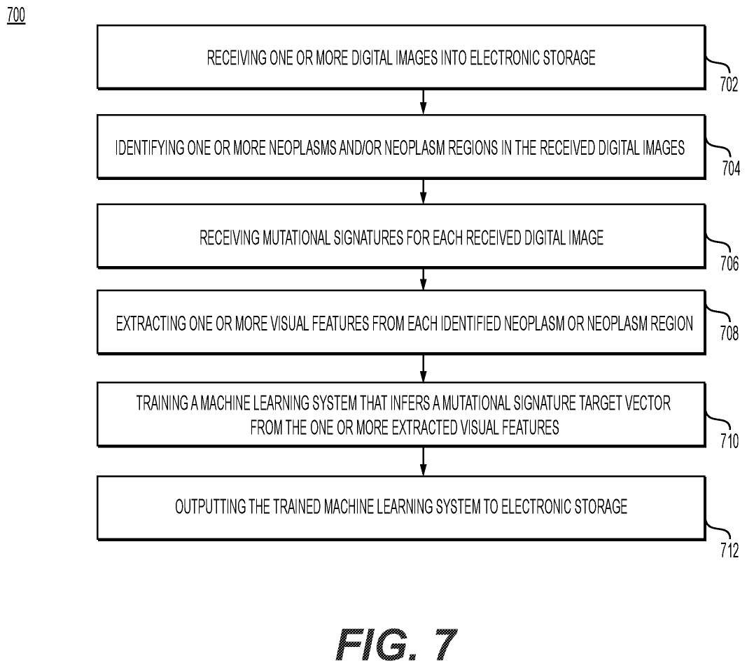

The data analysis module 433, neoplasm detection module 434, and/or the signature inference module 435 may analyze digitized images and determine whether a region in the sample needs further analysis. The identification of such may trigger an alert to a user. The neoplasm detection module 434 may identify or detect neoplasms, as described in more detail with reference to FIGS. 5-6. The signature inference module 435 may identify or determine signatures and/or tumor subtypes, as described in more detail with reference to FIGS. 6-7.

According to one embodiment, the target image platform 436 may include a target image intake module 436, a specimen detection module 437, and an output interface 438. The target image platform 436 may receive a target image and apply the machine learning model to the received target image to determine a characteristic of a target data set. For example, the target data may be received from any one or any combination of the server systems 410, physician servers 421, hospital servers 422, clinical trial servers 423, research lab servers 424, and/or laboratory information systems 425. The target image intake module 436 may receive a target dataset corresponding to a target health variable or a data variable. The specimen detection module 437 may apply the machine learning model to the target dataset to determine a characteristic of the target health variable or a data variable. For example, the specimen detection module 437 may detect a trend of the target relationship. The specimen detection module 437 may also apply the machine learning model to the target dataset to determine a quality score for the target dataset. Further, the specimen detection module 437 may apply the machine learning model to the target images to determine whether a target element is present in a determined relationship.

The output interface 438 may be used to output information about the target data and the determined relationship (e.g., to a screen, monitor, storage device, web browser, etc.). The output interface 438 may display identified salient regions of analyzed slides, detected neoplasms, detected signatures, tumor subtypes, etc. In some examples, the output interface 438 may output pie charts, bar graphs, lego charts, etc., such as those exemplified in FIGS. 1A through 1C.

### Neoplasm Detection Module 434

All tumors (e.g., cancer) are neoplasms, which are abnormal masses of tissue that form when cells grow and divide more than is typical or do not die when they should. Neoplasms may be benign or malignant (i.e., cancer), but it has been hypothesized that some benign neoplasms can later become malignant sub-clones of the original neoplasm.

The neoplasm detection module 434 may identify regions of a slide that have neoplasms. This identification may occur in a binary manner (neoplasm v. non-neoplasm) or alternatively may involve determining a kind of neoplasm in a region with a multi-class system (non-neoplasm v. neoplasm type 1 v. neoplasm type 2, etc.). For a multi-class approach, an example with breast cancer would be distinct outputs for invasive lobular carcinoma, invasive ductal carcinoma, ductal carcinoma in situ, lobular carcinoma in situ, atypical ductal hyperplasia, etc.

The neoplasm detection module 434 may be trained to identify, detect, or infer neoplasms for a specific tissue type, e.g., breast. Alternatively, the neoplasm detection module 434 may be trained to be pan-cancer so that it operates on multiple tissue types (breast, prostate, bladder, etc.).

Referring to FIG. 5, a method 500 for training the neoplasm detection module may include a step 502 of receiving one or more digital images (e.g., histology) into a digital or electronic storage device (e.g., hard drive, network drive, cloud storage, RAM, etc.). The method 500 may include a step 504 of receiving an indication of a presence and/or absence of any neoplasms. The method 500 may include a step 506 of receiving types of neoplasms present in each of the one or more digital images having neoplasms and/or receiving spatial locations of each neoplasm, which can be indicated with a binary pixel mask, a polygon, etc.

The method 500 may include a step 508 of breaking or segmenting each digital image into sub-regions. Regions may be specified in a variety of methods, including creating tiles of the image, segmentations based on edge/contrast, segmentations via color differences, segmentations based on energy minimization, supervised determination by the machine learning model, EdgeBoxes, SharpMask, etc.

The method 500 may include a step 510 of training a machine learning system that takes as input a digital image and infers the presence or absence of a neoplasm and/or the types and/or locations of neoplasms present. The method 500 may include a step 512 of saving and/or outputting the trained machine learning system to digital or electronic storage.

In step 510 including training, many methods could be used, including weak supervision, bounding box or polygon-based supervision, and/or Pixel-level or voxel-level labeling.

Weak supervision may include training a machine learning model (e.g., multi-layer perceptron or MLP, convolutional neural network or CNN, Transformers, graph neural network, support vector machine or SVM, random forest, etc.) using multiple instance learning (MIL) using weak labeling of a digital image or a collection of images. This approach may be used if a spatial location was not specified for an image.

Bounding box or polygon-based supervision may include training a machine learning model (e.g., region-based convolutional neural network or R-CNN, Faster R-CNN, Selective Search, etc.) using bounding boxes or polygons that specify sub-regions of the digital image.

Pixel-level or voxel-level labeling (e.g., a semantic or instance segmentation) may include training a machine learning model (e.g., Mask R-CNN, U-Net, Fully Convolutional Neural Network, Transformers, etc.) where individual pixels and/or voxels are identified as being neoplasms and/or a kind of neoplasm. Pixel-level and/or voxel-level labeling may be from a human annotator or may be from registered images.

Referring to FIG. 6, a method 600 for using the neoplasm detection module 434 may include a step 602 of receiving one or more digital images (e.g., medical or histology images) into a digital or electronic storage device (e.g., hard drive, network drive, cloud storage, RAM, etc.). The method 600 may include a step 604 of breaking or segmenting each digital image into sub-regions using an approach from training. The method 600 may include a step 606 of applying or running a trained machine learning system (e.g., the machine learning system trained using method 500) to each received digital image to infer or detect whether an image has neoplasms, which regions of the image are neoplasms, and/or a type of neoplasms present.

If neoplasms are determined to be present in step 606, step 606 may further include identifying spatial locations or regions of the neoplasms and flagging them. Detecting the regions may be done using a variety of methods, including running the machine learning model on image sub-regions to generate a prediction for each sub-region, and/or using machine learning visualization tools to create a detailed heatmap such as class activation maps, etc., and then extracting the relevant regions.

### Signature Inference Module 435

The signature inference module 435 may infer signatures present in a patient's neoplasms. Because tumors may be heterogeneous in that a patient may have tumor subclones or multiple tumors that arose independently, the signature inference module 435 may additionally produce spatial maps of the mutational signatures present.

Often, a tumor may have multiple signatures of varying levels. A signature may be treated as a percentage with each known signature being expressed at a given level. For example, a patient may have HRD as a highest signature, but other signatures may be simultaneously present at lower levels. To express levels of the signatures, signatures may be treated as proportions.

Referring to FIG. 7, a method 700 for training the signature inference module 435 may include a step 702 of receiving one or more digital images (e.g., medical images) into a digital or electronic storage device (e.g., hard drive, network drive, cloud storage, RAM, etc.) for one or more (e.g., a collection of) patients. The method 700 may include a step 704 of identifying one or more (e.g., all) neoplasms or neoplasm regions in the received digital images for each patient using, for example, the neoplasm detection module 434. Step 706 may include identifying a spatial location for each neoplasm.

The method 700 may include a step 706 of receiving mutational signatures for each received digital image into a digital or electronic storage device. This receiving of signatures and/or images may be done either at a patient level, image level, or local level (e.g., annotated pixels and/or voxels with the mutational signature). Signatures may be presented as a k-dimensional target vector that sums to 1, which may represent a proportion of each known signature or only a most present signature (i.e., a one-hot vector). Each element of this target vector may indicate a distinct signature, e.g., smoking.

The method 700 may include a step 708 of extracting one or more visual features (e.g., embeddings) from each identified neoplasm or neoplasm region. This extraction may be done with raw pixels/voxels or using a feature extractor for embeddings such as a convolutional neural network or a transformer which is trained with supervised or self-supervised learning. This extraction may transform visual information into a vector of features that represents each neoplasm.

The method 700 may include training a machine learning system that detects or infers a mutational signature target vector from the extracted visual features (e.g., neoplasm embeddings). Training may be done with any machine learning system, such as a random forest, a CNN trained with multiple instance learning, a vision transformer neural network, etc. For neural networks, inferring signature target vectors ma be done in multiple ways, including treating the signature target vectors as “soft targets” and training a network with cross-entropy loss with a softmax activation function that outputs a k-dimensional vector. The “soft target” approach may be a straightforward approach.

Step 710 may include training the machine learning system with open set recognition techniques to enable the machine learning system to suppress its outputs for unknown mutational signatures. This training may be done with approaches such as Tempered Mixup. This form of data augmentation may force the machine learning system to output a vector closer to a uniform distribution for unknown signatures so that all values may be as small as possible (e.g., near maximum entropy for unknown signatures).

The method 700 may include a step 712 of saving and/or outputting the trained machine learning system (e.g., learned parameters of a neural network of the trained machine learning system) to electronic storage.

A method 800 for using the signature inference module 435 may include a step 802 of receiving one or more digital images (e.g., medical images) into a digital storage device (e.g., hard drive, network drive, cloud storage, RAM, etc.) for a patient. The method 800 may include a step 804 identifying one or more (e.g., all) neoplasms or neoplasm regions on all receive images for the patient (for example, by using the neoplasm detection module 434). Step 804 may include a step of identifying all spatial locations of the identified neoplasms.

The method 800 may include a step 806 of extracting one or more visual features (e.g., embeddings) from each identified neoplasm or neoplasm regions. This extraction may be done with raw pixels and/or voxels or using a feature extractor for embeddings such as a convolutional neural network or a transformer which is trained with supervised or self-supervised learning. This extraction may transform visual information into a vector of features that represents each neoplasm.

The method 800 may include a step 808 of applying or running a trained machine learning system (such as a machine learning system trained using the method described with reference to FIG. 7) to detect or infer a mutational signature ratio vector for all of the extracted visual features (e.g., neoplasm embeddings). The method 800 may include a step 810 of determining whether a largest value in the mutational signature ratio vector is below a predetermined certainty threshold or a predetermined certainty (e.g., a real number between 0 and 1). If the largest value in the signature vector is determined to be below the certainty threshold, the method 800 may include a step 812 of indicating or outputting (e.g., flagging or notating) the patient and/or any associated neoplasms or regions as potentially having an unknown signature.

The method 800 may include a step 814 of performing an analysis and/or outputting the analysis (e.g., identified known signatures and new and/or unknown signatures) to electronic or digital storage. Step 804 may include producing an output overlay (e.g., a heatmap) on the image with each mutational signature identified, such as with color-coding. As explained hereinafter, step 814 may include outputting diagnoses, indications of cancer subtype, indications of subgroups or sets of patients with similar clinical phenotypes, decision trees for treatment options, a classification of patients as responders or non-responders for a certain therapy or treatment, and/or a likelihood of clinical benefit to a certain type of therapy. Some of these additional outputs may be based on further analysis by other modules in the disease detection platform 400. Methods 700 and/or 800 may be applied multiple times (e.g., before and after treatment or therapy) to detect changes in mutational signatures.

In addition, steps 802-812 may be repeated across multiple patients, and step 814 may include outputting, for any patient, neoplasm, or neoplasm region indicated as having an unknown signature in step 812, visual embeddings and/or patterns across these patients. Step 814 may include clustering these visual embeddings and/or patterns, e.g., using agglomerative clustering. Subsequently, the clustered patterns and/or embeddings may be correlated with a patient's clinical history and test information (including genomics testing) to identify whether any of these morphological signatures correspond to new signatures associated with unknown mutagens.

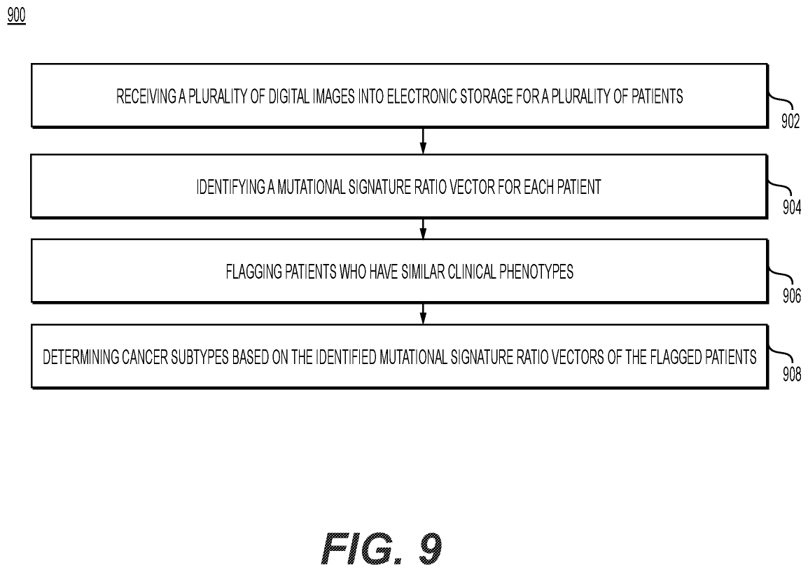

Step 814 may include any of the steps or processes explained with reference to FIGS. 9-14.

### Analysis of Signatures Based on Cancer Subtype

Cancer is a heterogenous disease that can develop in different tissues and cell types. Even within one cancer type, the disease may have multiple subtypes distinguished based on different histology and mutation patterns, which may lead to different clinical outcomes. Systems and methods disclosed herein may be used to build cancer subtype specific signatures. Other than common patient groups associated with signatures like HRD/smoking, systems and methods disclosed herein may identify new and rare tumor subtypes. Although prevalence may be low, with a computational pathology system used by hundreds of hospitals, such subgroups or sets of patients with similar clinical phenotypes (including similar mutational signature ratios within a predetermined threshold) may be flagged for effective therapeutic modalities.

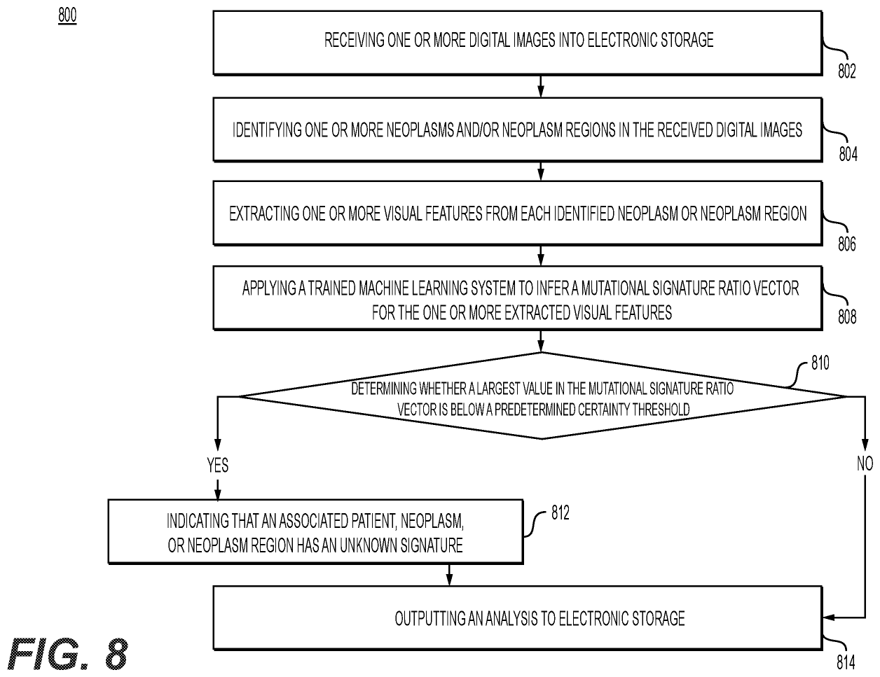

For example, referring to FIG. 9, a method 900 of determining a cancer subtype may include a step 902 of receiving a plurality of digital images for a plurality of patients into electronic storage. Step 902 may also include receiving patient information (e.g., clinical history, test information such as genomics testing, age, weight, diagnosis, medical history, clinical covariates, etc.) for each patient. The plurality of patients may have a same type of cancer, but perhaps different tumor, disease, or cancer subtypes. The method 900 may include a step 904 of identifying a mutational signature ratio vector for each patient (e.g., using the signature inference module 435 and/or the method 800 described with reference to FIG. 8). Alternatively, a mutational signature ratio vector may be identified for each digital image. The method 900 may include a step 906 of determining a set of and/or identifying patients who have similar clinical phenotypes (including similar mutational signatures), and a step 908 of determining disease (e.g., cancer) subtypes using the identified mutational signature ratio vectors of the flagged patients.

### Treatment Recommendation

Systems and methods disclosed herein may be used to provide treatment recommendations, such as by building decision trees for treatment options based on dominant signatures for cancer patients. For example, a composition of signatures may classify patients into two groups: responders to a certain type of therapy (e.g., neoadjuvant chemotherapy), and non-responders who may be spared side effects of the therapy (e.g., toxic side effects of chemotherapy). For a group of patients who are predicted to develop chemo-resistance, primary signatures of those patients may help further determine a likelihood of clinical benefit from immunotherapy or other targeted therapy.

Systems and methods disclosed herein may also trace changes in signatures pre- and post-treatment, predict patient drug responses in clinical trials, and reveal potential new targets to rehabilitate a response to chemo- or immuno-therapies for a chemo-immune-resistance phenotype.

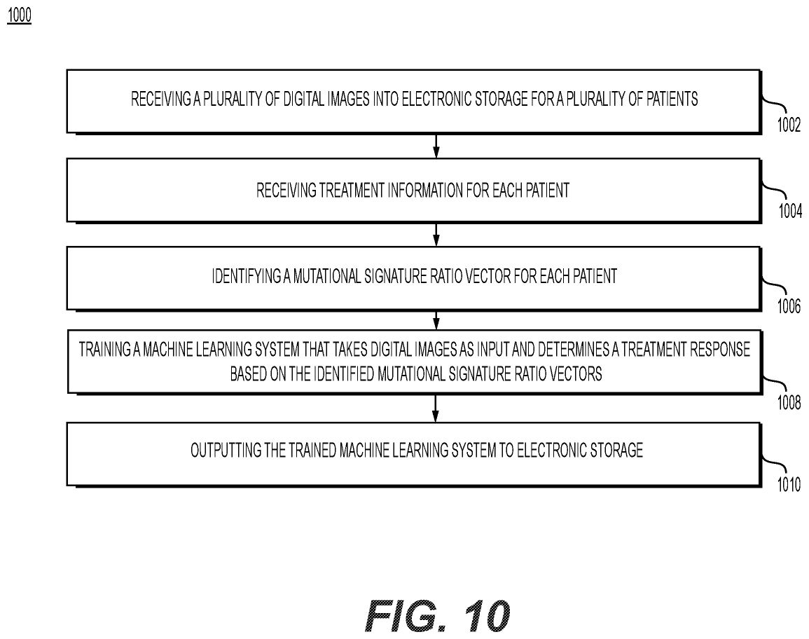

For example, referring to FIG. 10, a method 1000 of training a system to recommend treatment may include a step 1002 of receiving a plurality of digital images for a plurality of patients into electronic storage. Step 1002 may also include receiving patient information (e.g., clinical history, test information such as genomics testing, age, weight, diagnosis, medical history, clinical covariates, etc.) for each patient. The method 1000 may include a step 1004 of receiving treatment information for each patient. The treatment information may include how a patient responds to therapy or side effects. For example, the treatment information may include a treatment score correlating to how successful treatment was for the patient. As another example, the treatment information may include an indication of whether the patient responded to therapy or not. The method 1000 may include a step 1006 of identifying a mutational signature ratio vector for each patient (e.g., using the signature inference module 435 and/or the method 800 described with reference to FIG. 8). Alternatively, a mutational signature ratio vector may be identified for each digital image.

The method 1000 may include a step 1008 of training the system to take, as input, digital images and that determines a treatment response based on the identified mutational signature ratio vectors. The method 1000 may include a step 1010 of outputting the trained machine learning system to electronic storage. The method 1000 may also output (e.g., to a display) the learned relationships between mutational signature ratio vectors and treatment responses.

The system trained using method 1000 may be refined over time, For example, the method 1000 may repeat step 1004 by receiving update treatment information for certain patients. The system may be trained to predict drug responses or to determine a likelihood of immunotherapy.

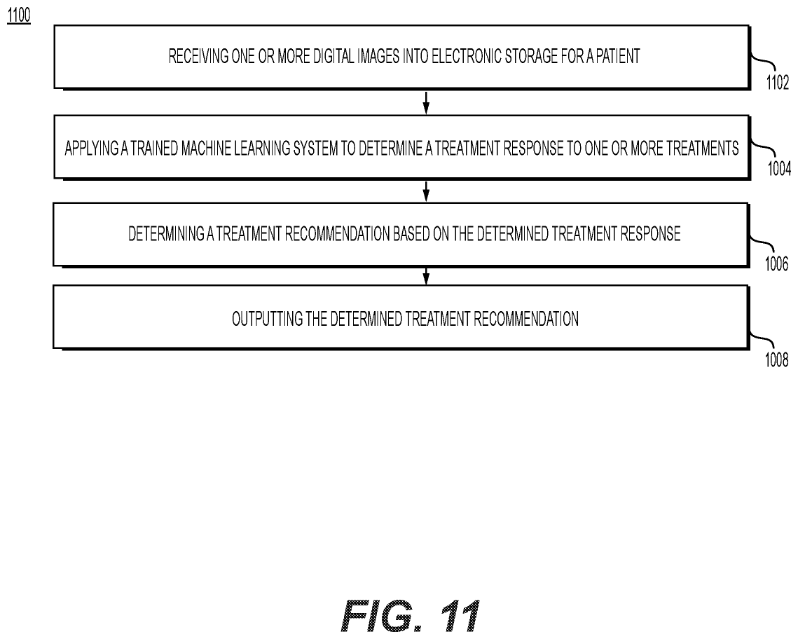

Referring to FIG. 11, a method 1100 of using such a system may include a step 1102 of receiving one or more digital images into electronic storage for a patient. Step 1102 may also include receiving patient information (e.g., clinical history, test information such as genomics testing, age, weight, diagnosis, medical history, clinical covariates, etc.) for the patient. The method 1100 may include a step 1104 of applying a trained system (e.g., the system trained using the method 1000 described with reference to FIG. 10) to determine a treatment response to one or more treatments. Step 1104 may include identifying a mutational signature ratio vector for each patient via the trained system and/or another trained system. The method 1000 may include a step 1006 of determining a treatment recommendation based on the determined treatment response and a step 1008 of outputting the treatment recommendation (e.g., to electronic storage and/or an electronic display). For example, where step 1004 included determining a treatment response for a plurality of treatments, step 1006 may include determining a best or most successful treatment among the plurality of treatment based on the determined treatment responses (e.g., based on determined treatment scores, a number of side effects, a severity of side effects or a side effect score, etc.) Where step 1004 included determining a treatment response for one treatment, step 1006 may include determining whether to recommend that treatment.

### Identifying New Mutational Signatures

Systems and methods disclosed herein may identify new mutational signatures. As previously explained, identification may be done by running systems disclosed herein with open set recognition capabilities on large datasets and identifying any neoplasms across patients that do not belong to any existing signatures. Subsequently, visual embeddings and/or patterns across these patients that are output from the neoplasm detection module 434 and identified by the signature inference module 435 as having an unknown signature may be clustered, e.g., using agglomerative clustering. Subsequently, the clustered patterns and/or embeddings may be correlated with a patient's clinical history and test information (including genomics testing) to identify whether any of these morphological signatures correspond to new signatures associated with unknown mutagens.

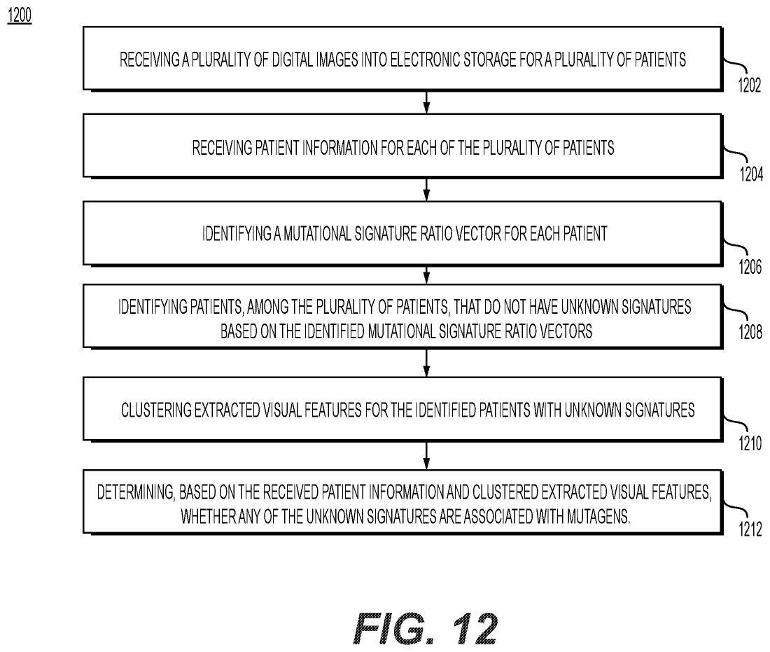

For example, referring to FIG. 12, a method 1200 of identifying new mutational signatures may include a step 1202 of receiving a plurality of digital images for a plurality of patients into electronic storage and a step 1204 of receiving patient information (e.g., clinical history, test information such as genomics testing, age, weight, diagnosis, medical history, clinical covariates, etc.) for each patient.

The method 1200 may include a step 1206 of identifying a mutational signature ratio vector for each patient (e.g., using the signature inference module 435 and/or the method 800 described with reference to FIG. 8). Alternatively, a mutational signature ratio vector may be identified for each digital image. As previously described, identifying mutational signature ratio vectors may include extracting one or more visual features from each identified neoplasm or neoplasm region.

The method 1200 may include a step 1208 of identifying patients, among the plurality of patients, that do not have any existing signatures and/or who have unknown signatures based on the identified mutational signature ratio vectors.

The method 1200 may include a step 1210 of clustering the extracted visual features for the identified patients with unknown signatures. The visual features may have been extracted in step 1206, or alternatively separately extracted. The visual features may include embeddings or patterns.

The method 1200 may include a step 1212 of determining, based on the received patient information and clustered extracted visual features, whether any of the unknown signatures are associated with mutagens. The mutagens may be unknown. Step 1212 may include, for example, recognizing similar features and/or patient characteristics for patients having same or similar mutational signature ratio vectors, and determining that those mutational signature ratio vectors may indicate a presence of an unknown mutagen.

### Identification of Mutagens

Many tumor signatures may be caused by known mutagens, e.g., smoking. However, many tumor signatures are not associated with known mutagens. Systems and methods disclosed herein may identify possible mutagens related to a tumor signature.

Current repositories of signatures still have unknowns regarding causes of the signatures. Systems and methods disclosed herein may mine data for new or old signatures and better correlate the signatures with clinical information about the patients to uncover risks of cancer that are not known in clinical covariates.

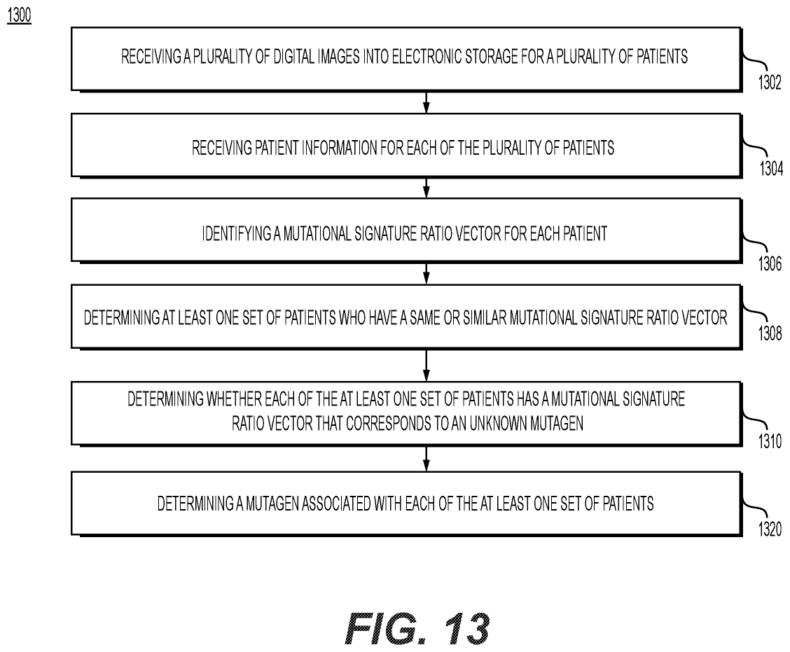

For example, referring to FIG. 13, a method 1300 of identifying unknown mutagens may be similar to a method 1200 of identifying unknown signatures. The method 1300 may include a step 1302 of receiving a plurality of digital images for a plurality of patients into electronic storage and a step 1304 of receiving patient information (e.g., clinical history, test information such as genomics testing, age, weight, diagnosis, medical history, clinical covariates, etc.) for each patient.

The method 1300 may include a step 1306 of identifying a mutational signature ratio vector for each patient (e.g., using the signature inference module 435 and/or the method 800 described with reference to FIG. 8). Alternatively, a mutational signature ratio vector may be identified for each digital image. As previously described, identifying mutational signature ratio vectors may include extracting one or more visual features from each identified neoplasm or neoplasm region.

The method 1300 may include a step 1308 of determining at least one set of patients who have a same or similar mutational signature ratio vector. Step 1308 may include clustering extracted features or determining similarities between extracted features for each patient.

The method 1300 may include a step 1310 of determining whether each of the at least one set of patients has a mutational signature ratio vector that corresponds to an unknown mutagen. The method 1300 may include a step 1320 of determining a mutagen associated with each of the at least one set of patients.

### Population Health Monitoring Based on Geography, Etc.

Systems and methods disclosed herein may build geographic location specific signatures. Systems and methods disclosed herein may help understand disease risk factors and how risks such as lifestyle, nature of environmental exposures, and genetics relate to the development of cancer. Systems and methods disclosed herein may support decision-making on expanding strategies for cancer prevention.

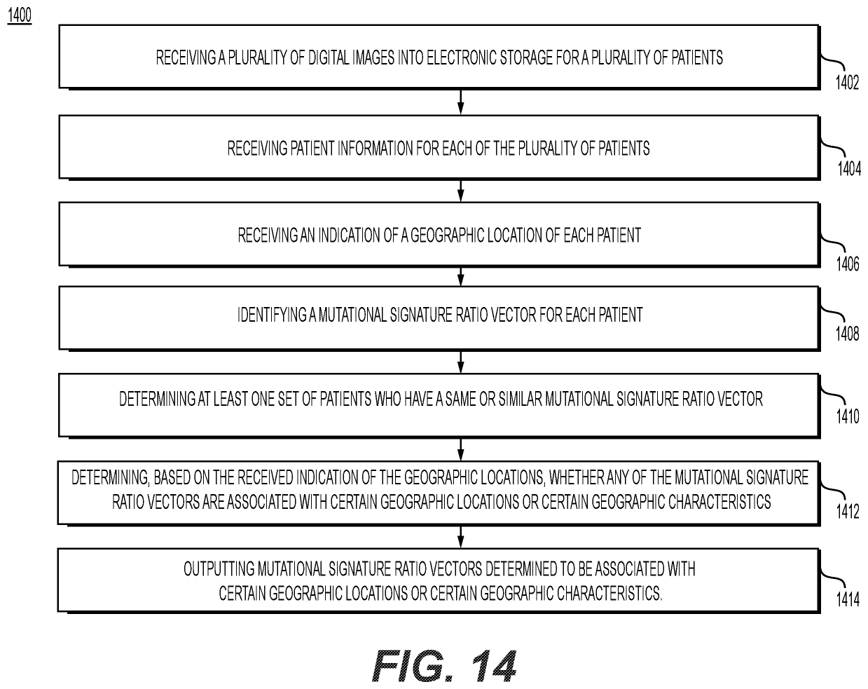

For example, referring to FIG. 14, a method 1400 of identifying a geographic location specific signature may be similar to method 1200. The method 1400 may include a step 1402 of receiving a plurality of digital images for a plurality of patients into electronic storage and a step 1404 of receiving patient information (e.g., clinical history, test information such as genomics testing, age, weight, diagnosis, medical history, clinical covariates, etc.) for each patient. The method 1400 may also include a step 1406 of receiving an indication of a geographic location of each patient. Step 1406 may also include receiving characteristics (e.g., weather, altitude, pollution level) etc. of each geographic location.

The method 1400 may include a step 1408 of identifying a mutational signature ratio vector for each patient (e.g., using the signature inference module 435 and/or the method 800 described with reference to FIG. 8). Alternatively, a mutational signature ratio vector may be identified for each digital image. As previously described, identifying mutational signature ratio vectors may include extracting one or more visual features from each identified neoplasm or neoplasm region.

The method 1400 may include a step 1408 of identifying patients, among the plurality of patients, that do not have any existing signatures and/or who have unknown signatures based on the identified mutational signature ratio vectors.

The method 1400 may include a step 1410 of determining at least one set of patients who have a same or similar mutational signature ratio vector. Step 1410 may include clustering extracted features or determining similarities between extracted features for each patient.

The method 1400 may include a step 1412 of determining, based on the received indication of the geographic locations, whether any of the mutational signature ratio vectors are associated with certain geographic locations or certain geographic characteristics. For example, step 1412 may include determining whether the set of patients who have a same or similar mutational signature ratio vector are associated with a same or similar geographic location or geographic characteristics. The method 1400 may include a step 1414 of outputting (e.g., to electronic storage and/or an electronic display) mutational signature ratio vectors determined to be associated with certain geographic locations or certain geographic characteristics.

Identifying Skin Cancers that Correspond to UV Morphological Mutation Signatures

Systems and methods disclosed herein may identify which skin cancers are related to ultraviolet (UV) exposure. Systems and methods disclosed herein may also identify other skin cancers that also correspond to UV morphological mutation signatures. For example, the methods 1200 and/or 1300 described with reference to FIGS. 12 and 13 may be used to determine relationships between certain skin cancers, certain subtypes of cancer, etc. and certain types or lengths of UV exposure or exposure to other substances.

### Predict Cancer Metastasis

Metastasis patterns in cancer vary both spatially and temporally. Systems and methods disclosed herein may trace whether there are new signatures that evolve across time (and/or how these new signatures evolve), create a spatial map of mutational signatures, detect clonal expansion, and further predict cancer metastasis. For example, the methods 1200 described with reference to FIG. 12 may be used to determine new signatures and/or track signatures over time as more information is received in step 1204 and/or determine relationships between cancer metastasis and identified signatures and/or changes. In addition, the method 1200 (and/or any of methods 800 through 1400) may include outputting spatial maps of mutational signatures (e.g., as identified in steps 1206 and/or 1212) detected changes, clonal expansions, predictions of a disease (e.g., cancer metastasis), etc.

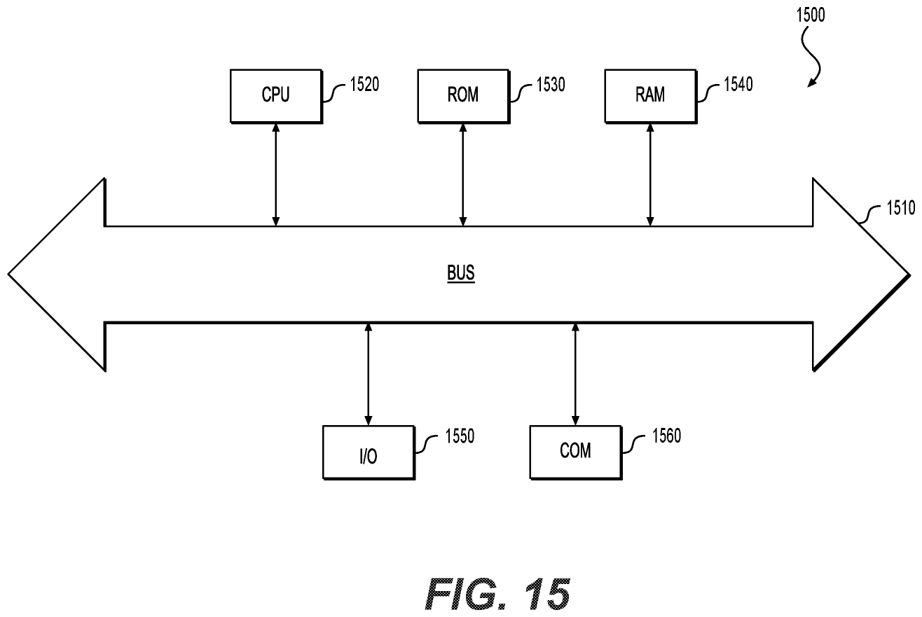

Referring to FIG. 15, a device 1500 may include a central processing unit (CPU) 1520. CPU 1520 may be any type of processing device including, for example, any type of special purpose or a general-purpose microprocessor device. As will be appreciated by persons skilled in the relevant art, CPU 1520 also may be a single processor in a multi-core/multiprocessor system, such system operating alone, or in a cluster of computing devices operating in a cluster or server farm. CPU 1520 may be connected to a data communication infrastructure 1510, for example a bus, message queue, network, or multi-core message-passing scheme.

Device 1500 may also include a main memory 1540, for example, random access memory (RAM), and may also include a secondary memory 1530. Secondary memory 1530, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 1530 may include similar means for allowing computer programs or other instructions to be loaded into device 1500. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 1500.

Device 1500 also may include a communications interface (“COM”) 1560. Communications interface 1560 allows software and data to be transferred between device 1500 and external devices. Communications interface 1560 may include a model, a network interface (such as an Ethernet card), a communications, a PCMCIA slot and card, or the like. Software and data transferred via communications interface 1560 may be in the form of signals, which may be electronic, electromagnetic, optical or other signals capable of being received by communications interface 1560. These signals may be provided to communications interface 1560 via a communications path of device 1500, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 1500 may also include input and output ports 1550 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other embodiments of the invention may be apparent to those skilled in the art from consideration of the specification and practice of the invention disclosed herein. It is intended that the specification and examples be considered as exemplary only.

What is claimed is:

1. A computer-implemented method for identifying a mutational signature, comprising:
receiving one or more digital images into electronic storage for at least one patient;
identifying one or more neoplasms in each received digital image;
extracting one or more visual features from each identified neoplasm; and
applying a trained machine learning system to identify a mutational signature ratio vector for the one or more extracted visual features.

2. The method of claim 1, wherein the extracted visual features are neoplasm embeddings.

3. The method of claim 1, wherein identifying one or more neoplasms includes segmenting each received digital image into subregions.

4. The method of claim 1, the method further comprises determining, for each identified mutational signature ratio vector, whether a largest value in the mutational signature ratio vector is below a predetermined certainty threshold.

5. The method of claim 4, wherein the method further comprises determining that the largest value in the mutational signature ratio is below the predetermined certainty threshold, and determining that a mutational signature corresponding to the mutational signature ratio is unknown.

6. The method of claim 5, wherein receiving one or more digital images for at least one patient includes receiving a plurality of digital images for a plurality of patients, wherein applying the trained machine learning system to identify the mutational signature ratio vector includes identifying a plurality of mutational signature ratio vectors, and wherein the method further comprises identifying a set of patients among the plurality of patients that have an unknown mutational signature.

7. The method of claim 6, further comprising clustering extracted visual features for the identified set of patients.

8. The method of claim 7, further comprising:
receiving patient information for each patient among the plurality of patients; and
determining, based on the received patient information and clustered extracted visual features, whether any of the unknown signatures are associated with mutagens.

9. The method of claim 1, wherein receiving one or more digital images for at least one patient includes receiving a plurality of digital images for a plurality of patients, wherein applying the trained machine learning system to identify the mutational signature ratio vector includes identifying a plurality of mutational signature ratio vectors, and the method further includes:
receiving patient information for each patient among the plurality of patients;
determining a set of patients who have similar clinical phenotypes; and
determining disease subtypes based on the identified mutational signature ratio vectors of the determined set of patients.

10. The method of claim 1, wherein receiving one or more digital images for at least one patient includes receiving a plurality of digital images for a plurality of patients, wherein applying the trained machine learning system to identify the mutational signature ratio vector includes identifying a plurality of mutational signature ratio vectors, and the method further includes:
receiving treatment information for each patient among the plurality of patients; and
training a machine learning system that predicts a treatment response based on the identified mutational signature ratio vectors and received treatment information.

11. The method of claim 1, wherein receiving one or more digital images for at least one patient includes receiving a plurality of digital images for a plurality of patients, wherein applying the trained machine learning system to identify the mutational signature ratio vector includes identifying a plurality of mutational signature ratio vectors, and the method further includes:
receiving patient information for each of the plurality of patients;
receiving an indication of a geographic location of each patient; and
determining, based on the received indications of the geographic locations, whether any of the mutational signature ratio vectors are associated with certain geographic locations.

12. The method of claim 1, wherein receiving one or more digital images for at least one patient includes receiving a plurality of digital images for a plurality of patients, wherein applying the trained machine learning system to identify the mutational signature ratio vector includes identifying a plurality of mutational signature ratio vectors, and wherein the method further comprises:
clustering extracted visual features for the plurality of patients; and
determining a mutational signature ratio vector among the identified mutational signature ratio vectors correspond to an unknown mutagen.

13. A system for processing electronic medical images, the system comprising:
at least one memory storing instructions; and
at least one processor configured to execute the instructions to perform operations comprising:
receiving one or more digital images into electronic storage for at least one patient;
identifying one or more neoplasms in each received digital image;
extracting one or more visual features from each identified neoplasm; and
applying a trained machine learning system to identify a mutational signature ratio vector for the one or more extracted visual features.

14. The system of claim 13, wherein the operations further comprise determining, for each identified mutational signature ratio vector, whether a largest value in the mutational signature ratio vector is below a predetermined certainty threshold.

15. The system of claim 14, wherein the operations further comprise determining that the largest value in the mutational signature ratio is below the predetermined certainty threshold, and determining that a mutational signature corresponding to the mutational signature ratio is unknown.

16. The system of claim 15, wherein receiving one or more digital images for at least one patient includes receiving a plurality of digital images for a plurality of patients, wherein applying the trained machine learning system to identify the mutational signature ratio vector includes identifying a plurality of mutational signature ratio vectors, and wherein the operations further comprise identifying a set of patients among the plurality of patients that have an unknown mutational signature.

17. The system of claim 16, wherein the operations further comprise clustering extracted visual features for the identified set of patients.

18. The system of claim 17, wherein the operations further comprise:
receiving patient information for each patient among the plurality of patients; and
determining, based on the received patient information and clustered extracted visual features, whether any of the unknown signatures are associated with mutagens.

19. A non-transitory computer-readable medium storing instructions that, when executed by a processor, perform operations processing electronic medical images, the operations comprising:
receiving one or more digital images into electronic storage for at least one patient;
identifying one or more neoplasms in each received digital image;
extracting one or more visual features from each identified neoplasm; and
applying a trained machine learning system to identify a mutational signature ratio vector for the one or more extracted visual features.

20. The computer-readable medium of claim 19, wherein the operations further comprise determining, for each identified mutational signature ratio vector, whether a largest value in the mutational signature ratio vector is below a predetermined certainty threshold.
